SEQUENCE LISTING (12 sequences)

SEQ ID NO: 1 - DNA, 2228 bp, Homo sapiens
cttaagagcgcggccggaaagcagttgagttacagacatcctgccaaaatgatttcttcaaagcccagacttgtcgtaccctatggcctcaagactctgctcgagggaattagcagagctgttctcaaaaccaacccatcaaacatcaaccagtttgcagcagcttattttcaagaacttactatgtatagagggaatactactatggatataaaagatctggttaaacaatttcatcagattaaagtagagaaatggtcagaaggaacgacaccacagaagaaattagaatgtttaaaagaaccaggaaaaacatctgtagaatctaaagtacctacccagatggaaaaatctacagacacagacgaggacaatgtaaccagaacagaatatagtgacaaaaccacccagtttccatcagtttatgctgtgccaggcactgagcaaacggaagcagttggtggtctttcttccaaaccagccacccctaagactactaccccaccctcatcaccacctccaacagctgtctcaccagagtttgcctacgtcccagctgacccagctcagcttgctgctcagatgttaggtaaagtttcatctattcattctgatcaatctgatgtgttaatggtggatgtggcaaccagtatgcctgttgttatcaaggaggtgccaagctcagaggctgctgaagatgtcatggtggctgctcctcttgtgtgttctggaaaggtgctagaagtgcaggttgtgaaccaaacatctgtccatgtagatttgggttctcaacctaaagaaaatgaggctgaaccatcaacggcttcctcagtccccttgcaggatgaacaagaacctcctgcttatgatcaagctcctgaggtcactttgcaggctgatattgaggttatgtcaactgttcatatatcatctgtctataacgatgtgcctgtgactgaaggagttgtttatatcgagcaactgccagaacaaatagttatcccttttactgatcaagttgcttgtcttaaagaaaatgagcagtcaaaagaaaatgagcagtcaccacgagttagtcccaaatctgtagtagaaaagaccacctctggcatgtctaaaaaatctgtagagtccgtaaaacttgcacagttggaggagaatgcaaaatattcctcagtatatatggaggcagaagcaacagctctgctctctgacacatctttgaaaggtcagcctgaggtacctgcacaactcctggatgcagaaggtgctatcaaaataggctctgaaaaatctctgcaccttgaagtggaggtcacttcaatagtctctgacaatactgggcaggaggagtctggggaaaactctgtaccccaggagatggaaggcagacctgtgctctctggggaagctgcagaagcagtgcactcaggtacatctgtaaagtcatctagtggccccttccctcctgctccagaaggccttactgcaccagaaattgaaccagaaggggaatcaacagctgaataaggtttgatgaagccagcaatggcaacaagtgaacgaggacaaccaccaccatgttctaacatgtggaccctttattgtctaactgataagaatcaacaaggtcacccatcaccgccacctgcacctgggccttttccccaagcaaccctctatttacctaatcctaaggatccacagtttcagcagcatccaccaaaagtcacttttccaacttatgtgatgggcgacaccaagaagaccagtgccccaccttttatcttagtaggctcaaatgttcaggaagcacagggatggaaacctcttcccggacatgctgtcgtttcacagtcagatgtcttgagatatgttgcaatgcaagtgcccattgctgttcctgcagatgagaaataccagaaacataccctaagtccccagaatgctaatcctccaagtggacaagatgtccccaggccaaaaagccctgttttcctttctgttgctttcccagtagaagatgtagctaaaaaaagttcaggatctggtgacaaatgtgctccctttggaagttacggtattgctggggaggtaaccgtgactactgctcacaaacgtcgcaaagcagaaactgaaaactgatccagaaatgacgctgtctgggtcaacatttcagggaggagtctgccaccagtgtaatgtatcaataaacttcatgcaagctt

SEQ ID NO: 2 - protein, 493 aa, Homo sapiens
MISSKPRLVVPYGLKTLLEGISRAVLKTNPSNINQFAAAYFQELTMYRGNTTMDIKDLVKQFHQIKVEKWSEGTTPQKKLECLKEPGKTSVESKVPTQMEKSTDTDEDNVTRTEYSDKTTQFPSVYAVPGTEQTEAVGGLSSKPATPKTTTPPSSPPPTAVSPEFAYVPADPAQLAAQMLGKVSSIHSDQSDVLMVDVATSMPVVIKEVPSSEAAEDVMVAAPLVCSGKVLEVQVVNQTSVHVDLGSQPKENEAEPSTASSVPLQDEQEPPAYDQAPEVTLQADIEVMSTVHISSVYNDVPVTEGVVYIEQLPEQIVIPFTDQVACLKENEQSKENEQSPRVSPKSVVEKTTSGMSKKSVESVKLAQLEENAKYSSVYMEAEATALLSDTSLKGQPEVPAQLLDAEGAIKIGSEKSLHLEVEVTSIVSDNTGQEESGENSVPQEMEGRPVLSGEAAEAVHSGTSVKSSSGPFPPAPEGLTAPEIEPEGESTAE

SEQ ID NO: 3 - DNA, 2174 bp, Homo sapiens
cttaagagcgcggccggaaagcagttgagttacagacatcctgccaaaatgatttcttcaaagcccagacttgtcgtaccctatggcctcaagactctgctcgagggaattagcagagctgttctcaaaaccaacccatcaaacatcaaccagtttgcagcagcttattttcaagaacttactatgtatagagtagagaaatggtcagaaggaacgacaccacagaagaaattagaatgtttaaaagaaccaggaaaaacatctgtagaatctaaagtacctacccagatggaaaaatctacagacacagacgaggacaatgtaaccagaacagaatatagtgacaaaaccacccagtttccatcagtttatgctgtgccaggcactgagcaaacggaagcagttggtggtctttcttccaaaccagccacccctaagactactaccccaccctcatcaccacctccaacagctgtctcaccagagtttgcctacgtcccagctgacccagctcagcttgctgctcagatgttaggtaaagtttcatctattcattctgatcaatctgatgtgttaatggtggatgtggcaaccagtatgcctgttgttatcaaggaggtgccaagctcagaggctgctgaagatgtcatggtggctgctcctcttgtgtgttctggaaaggtgctagaagtgcaggttgtgaaccaaacatctgtccatgtagatttgggttctcaacctaaagaaaatgaggctgaaccatcaacggcttcctcagtccccttgcaggatgaacaagaacctcctgcttatgatcaagctcctgaggtcactttgcaggctgatattgaggttatgtcaactgttcatatatcatctgtctataacgatgtgcctgtgactgaaggagttgtttatatcgagcaactgccagaacaaatagttatcccttttactgatcaagttgcttgtcttaaagaaaatgagcagtcaaaagaaaatgagcagtcaccacgagttagtcccaaatctgtagtagaaaagaccacctctggcatgtctaaaaaatctgtagagtccgtaaaacttgcacagttggaggagaatgcaaaatattcctcagtatatatggaggcagaagcaacagctctgctctctgacacatctttgaaaggtcagcctgaggtacctgcacaactcctggatgcagaaggtgctatcaaaataggctctgaaaaatctctgcaccttgaagtggaggtcacttcaatagtctctgacaatactgggcaggaggagtctggggaaaactctgtaccccaggagatggaaggcagacctgtgctctctggggaagctgcagaagcagtgcactcaggtacatctgtaaagtcatctagtggccccttccctcctgctccagaaggccttactgcaccagaaattgaaccagaaggggaatcaacagctgaataaggtttgatgaagccagcaatggcaacaagtgaacgaggacaaccaccaccatgttctaacatgtggaccctttattgtctaactgataagaatcaacaaggtcacccatcaccgccacctgcacctgggccttttccccaagcaaccctctatttacctaatcctaaggatccacagtttcagcagcatccaccaaaagtcacttttccaacttatgtgatgggcgacaccaagaagaccagtgccccaccttttatcttagtaggctcaaatgttcaggaagcacagggatggaaacctcttcccggacatgctgtcgtttcacagtcagatgtcttgagatatgttgcaatgcaagtgcccattgctgttcctgcagatgagaaataccagaaacataccctaagtccccagaatgctaatcctccaagtggacaagatgtccccaggccaaaaagccctgttttcctttctgttgctttcccagtagaagatgtagctaaaaaaagttcaggatctggtgacaaatgtgctccctttggaagttacggtattgctggggaggtaaccgtgactactgctcacaaacgtcgcaaagcagaaactgaaaactgatccagaaatgacgctgtctgggtcaacatttcagggaggagtctgccaccagtgtaatgtatcaataaacttcatgcaagctt

SEQ ID NO: 4 - protein, 475 aa, Homo sapiens
MISSKPRLVVPYGLKTLLEGISRAVLKTNPSNINQFAAAYFQELTMYRVEKWSEGTTPQKKLECLKEPGKTSVESKVPTQMEKSTDTDEDNVTRTEYSDKTTQFPSVYAVPGTEQTEAVGGLSSKPATPKTTTPPSSPPPTAVSPEFAYVPADPAQLAAQMLGKVSSIHSDQSDVLMVDVATSMPVVIKEVPSSEAAEDVMVAAPLVCSGKVLEVQVVNQTSVHVDLGSQPKENEAEPSTASSVPLQDEQEPPAYDQAPEVTLQADIEVMSTVHISSVYNDVPVTEGVVYIEQLPEQIVIPFTDQVACLKENEQSKENEQSPRVSPKSVVEKTTSGMSKKSVESVKLAQLEENAKYSSVYMEAEATALLSDTSLKGQPEVPAQLLDAEGAIKIGSEKSLHLEVEVTSIVSDNTGQEESGENSVPQEMEGRPVLSGEAAEAVHSGTSVKSSSGPFPPAPEGLTAPEIEPEGESTAE

SEQ ID NO: 5 - DNA, 1272 bp, Homo sapiens
cccatggctccgaagtccccgctggcatcctgaaagttgagttacagacatcctgccaaaatgatttcttcaaagcccagacttgtcgtaccctatggcctcaagactctgctcgagggaattagcagagctgttctcaaaaccaacccatcaaacatcaaccagtttgcagcagcttattttcaagaacttactatgtatagagggaatactactatggatataaaagatctggttaaacaatttcatcagattaaagtagagaaatggtcagaaggaacgacaccacagaagaaattagaatgtttaaaagaaccaggaaaaacatctgtagaatctaaagtacctacccagatggaaaaatctacagacacagacgaggacaatgtaaccagaacagaatatagtgacaaaaccacccagtttccatcagtttatgctgtgccaggcactgagcaaacggaagcagttggtggtctttcttccaaaccagccacccctaagactactaccccaccctcatcaccacctccaacagctgtctcaccagagtttgcctacgtcccagctgacccagctcagcttgctgctcagatgttagcaatggcaacaagtgaacgaggacaaccaccaccatgttctaacatgtggaccctttattgtctaactgataagaatcaacaaggtcacccatcaccgccacctgcacctgggccttttccccaagcaaccctctatttacctaatcctaaggatccacagtttcagcagcatccaccaaaagtcacttttccaacttatgtgatgggcgacaccaagaagaccagtgccccaccttttatcttagtaggctcaaatgttcaggaagcacagggatggaaacctcttcccggacatgctgtcgtttcacagtcagatgtcttgagatatgttgcaatgcaagtgcccattgctgttcctgcagatgagaaataccagaaacataccctaagtccccagaatgctaatcctccaagtggacaagatgtccccaggccaaaaagccctgttttcctttctgttgctttcccagtagaagatgtagctaaaaaaagttcaggatctggtgacaaatgtgctccctttggaagttacggtattgctggggaggtaaccgtgactactgctcacaaacgtcgcaaagcagaaactgaaaactgatccagaaatgacgctgtctgggtcaacatttcagggaggagtctgccaccagtgtaatgtatcaataaactt

SEQ ID NO: 6 - protein, 379 aa, Homo sapiens
MISSKPRLVVPYGLKTLLEGISRAVLKTNPSNINQFAAAYFQELTMYRGNTTMDIKDLVKQFHQIKVEKWSEGTTPQKKLECLKEPGKTSVESKVPTQMEKSTDTDEDNVTRTEYSDKTTQFPSVYAVPGTEQTEAVGGLSSKPATPKTTTPPSSPPPTAVSPEFAYVPADPAQLAAQMLAMATSERGQPPPCSNMWTLYCLTDKNQQGHPSPPPAPGPFPQATLYLPNPKDPQFQQHPPKVTFPTYVMGDTKKTSAPPFILVGSNVQEAQGWKPLPGHAVVSQSDVLRYVAMQVPIAVPADEKYQKHTLSPQNANPPSGQDVPRPKSPVFLSVAFPVEDVAKKSSGSGDKCAPFGSYGIAGEVTVTTAHKRRKAETEN

SEQ ID NO: 7 - DNA, 1089 bp, Homo sapiens
atgcctgttgttatcaaggaggtgccaagctcagaggctgctgaagatgtcatggtggctgctcctcttgtgtgttctggaaaggtgctagaagtgcaggttgtgaaccaaacatctgtccatgtagatttgggttctcaacctaaagaaaatgaggctgaaccatcaacggcttcctcagtccccttgcaggatgaacaagaacctcctgcttatgatcaagctcctgaggtcactttgcaggctgatattgaggttatgtcaactgttcatatatcatctgtctataacgatgtgcctgtgactgaaggagttgtttatatcgagcaactgccagaacaaatagttatcccttttactgatcaagttgcttgtcttaaagaaaatgagcagtcaaaagaaaatgagcagtcaccacgagttagtcccaaatctgtagtagaaaagaccacctctggcatgtctaaaaaatctgtagagtctgtaaaacttgcacagttggaggagaatgcaaaatattcctcagtatatatggaggcagaagcaacagctctgctctctgacacatctttgaaaggtcagcctgaggtacctgcacaactcctggatgcagaaggtgctatcaaaataggctctgaaaaatctctgcaccttgaagtggagatcacttcaatagtctctgacaatactgggcaggaggagtctggggaaaactctgtaccccaggagatggaaggcaaacctgtgctctctggggaagctgcagaagcagtgcactcaggtacatctgtaaagtcatctagtggccccttccctcctgctccagaaggccttactgcaccagaaattgaaccagaaggggaagcaacagctgaataaggtttgatgaagccagtagaagatgtagctaaaaaaagttcaggatctggtgacaaatgtgctccctttggaagttacggtattgctggggaggtaaccgtgactactgctcacaaacgtcgcaaagcagaaactgaaaactgatccagaaatgacgctgtctgggtcaacatttcagggaggagtctgccaccagtgtaatgtatcaat

SEQ ID NO: 8 - protein, 292 aa, Homo sapiens
MPVVIKEVPSSEAAEDVMVAAPLVCSGKVLEVQVVNQTSVHVDLGSQPKENEAEPSTASSVPLQDEQEPPAYDQAPEVTLQADIEVMSTVHISSVYNDVPVTEGVVYIEQLPEQIVIPFTDQVACLKENEQSKENEQSPRVSPKSVVEKTTSGMSKKSVESVKLAQLEENAKYSSVYMEAEATALLSDTSLKGQPEVPAQLLDAEGAIKIGSEKSLHLEVEITSIVSDNTGQEESGENSVPQEMEGKPVLSGEAAEAVHSGTSVKSSSGPFPPAPEGLTAPEIEPEGEATAE

SEQ ID NO: 9 - DNA, 1044 bp, Homo sapiens
taagagcgcggccggaaagcagtagagaaatggtcagaaggaacgacaccacagaagaaattagaatgtttaaaagaaccaggaaaaacatctgtagaatctaaagtacctacccagatggaaaaatctacagacacagacgaggacaatgtaaccagaacagaatatagtgacaaaaccacccagtttccatcagtttatgctgtgccaggcactgagcaaacggaagcagttggtggtctttcttccaaaccagccacccctaagactactaccccaccctcatcaccacctccaacagctgtctcaccagagtttgcctacgtcccagctgacccagctcagcttgctgctcagatgttagcaatggcaacaagtgaacgaggacaaccaccaccatgttctaacatgtggaccctttattgtctaactgataagaatcaacaaggtcacccatcaccgccacctgcacctgggccttttccccaagcaaccctctatttacctaatcctaaggatccacagtttcagcagcatccaccaaaagtcacttttccaacttatgtgatgggcgacaccaagaagaccagtgccccaccttttatcttagtaggctcaaatgttcaggaagcacagggatggaaacctcttcccggacatgctgtcgtttcacagtcagatgtcttgagatatgttgcaatgcaagtgcccattgctgttcctgcagatgagaaataccagaaacataccctaagtccccagaatgctaatcctccaagtggacaagatgtccccaggccaaaaagccctgttttcctttctgttgctttcccagtagaagatgtagctaaaaaaagttcaggatctggtgacaaatgtgctccctttggaagttacggtattgctggggaggtaaccgtgactactgctcacaaacgtcgcaaagcagaaactgaaaactgatccagaaatgacgctgtctgggtcaacatttcagggaggagtctgccaccagtgtaatgtatcaataaacttcatgcaagc

SEQ ID NO: 10 - protein, 281 aa, Homo sapiens
MEKSTDTDEDNVTRTEYSDKTTQFPSVYAVPGTEQTEAVGGLSSKPATPKTTTPPSSPPPTAVSPEFAYVPADPAQLAAQMLAMATSERGQPPPCSNMWTLYCLTDKNQQGHPSPPPAPGPFPQATLYLPNPKDPQFQQHPPKVTFPTYVMGDTKKTSAPPFILVGSNVQEAQGWKPLPGHAVVSQSDVLRYVAMQVPIAVPADEKYQKHTLSPQNANPPSGQDVPRPKSPVFLSVAFPVEDVAKKSSGSGDKCAPFGSYGIAGEVTVTTAHKRRKAETEN

SEQ ID NO: 11 - DNA, 792 bp, Homo sapiens
cttaagagcgcggccggaaagcagttgagttacagacatcctgccaaaatgatttcttcaaagcccagacttgtcgtaccctatggcctcaagactctgctcgagggaattagcagagctgttctcaaaaccaacccatcaaacatcaaccagtttgcagcagcttattttcaagaacttactatgtatagagggaatactactatggatataaaagatctggttaaacaatttcatcagattaaagtagagaaatggtcagaaggaacgacaccacagaagaaattagaatgtttaaaagaaccaggaaaaacatctgtagaatctaaagtacctacccagatggaaaaatctacagacacagacgaggacaatgtaaccagaacagaatatagtgacaaaaccacccagtttccatcagtttatgctgtgccaggcactgagcaaacggaagcagttggtggtctttcttccaaaccagccacccctaagactactaccccaccctcatcaccacctccaacagctgtctcaccagagtttgcctacgtcccagctgacccagctcagcttgctgctcagatgttagaagatgtagctaaaaaaagttcaggatctggtgacaaatgtgctccctttggaagttacggtattgctggggaggtaaccgtgactactgctcacaaacgtcgcaaagcagaaactgaaaactgatccagaaatgacgctgtctgggtcaacatttcagggaggagtctgccaccagtgtaatgtatcaataaacttcatgca

SEQ ID NO: 12 - protein, 221 aa, Homo sapiens
MISSKPRLVVPYGLKTLLEGISRAVLKTNPSNINQFAAAYFQELTMYRGNTTMDIKDLVKQFHQIKVEKWSEGTTPQKKLECLKEPGKTSVESKVPTQMEKSTDTDEDNVTRTEYSDKTTQFPSVYAVPGTEQTEAVGGLSSKPATPKTTTPPSSPPPTAVSPEFAYVPADPAQLAAQMLEDVAKKSSGSGDKCAPFGSYGIAGEVTVTTAHKRRKAETEN

(19) United States
(12) Patent Application Publication
(10) Pub. No.: US 2009/0136962 A1
(43) Pub. Date: May 28, 2009

(54) DIAGNOSTIC MARKERS FOR CANCER

(76) Inventors: John C. HERR, Charlottesville, VA (US); Arabinda MANDAL, Charlottesville, VA (US); Henry F. FRIERSON, Jr., Charlottesville, VA (US)

Correspondence Address: BARNES & THORNBURG LLP, 11 SOUTH MERIDIAN, INDIANAPOLIS, IN, 46204, US

(21) Appl. No.: 12/323,541

(22) Filed: Nov. 26, 2008

Related U.S. Application Data
(60) Provisional application No. 60/990,317, filed on Nov. 27, 2007.
(60) Provisional application No. 61/109,542, filed on Oct. 30, 2008.

(51) Int. Cl. C12Q 1/68; G01N 33/68; G01N 33/536; C07K 16/18; C07K 19/00

(52) U.S. Cl. 435/6; 436/86; 436/536; 530/387.1; 530/387.3; 530/388.2; 530/391.7

(57) ABSTRACT

The present invention provides compositions and methods useful for diagnosing cancer and for monitoring the progression and treatment of cancer, as well as targets for treating cancer. The invention provides sperm and testis associated antigens, such as the protein CABYR, as biomarkers and as targets for chemotherapy.

### CROSS-REFERENCE TO RELATED APPLICATIONS

This application claims the benefit of U.S. Provisional Application Nos. 60/990,317, filed Nov. 27, 2007 and 61/109,542, filed Oct. 30, 2008, which are incorporated herein by reference in their entirety.

### STATEMENT REGARDING FEDERALLY SPONSORED RESEARCH OR DEVELOPMENT

This invention was made in part with United States Government support under National Institutes of Health Grant Nos. T32 HD07382, T32 DK07642, D43TW000654-14 (Fogarty International Center) and U54 29099, National Institute of Justice No. 2000-IJ-CX-K013, and Federal Bureau of Investigations No. 115744. The United States Government has certain rights in the invention.

### BACKGROUND

The flagellar protein CABYR (a.k.a. calcium binding protein 86 or fibrosheathin 2) is encoded by a single copy gene and represents a calcium binding protein that is tyrosine phosphorylation-regulated during capacitation. Sequences encoding CABYR were cloned and characterized in human and mouse sperm (Naaby-Hansen et al., 2002, Dev. Biol., 242:236-254; Buer et al., 2003, Gene, 310:67-78). CABYR mRNAs were found to be abundantly expressed in the testis and translated post-meiotically only in round spermatids. In humans, six alternative splice variants of CABYR were discovered which are all translated in spermatids resulting in considerable CABYR protein microheterogeneity. CABYR is localized to the ribs and longitudinal columns of the fibrous sheath, a cytoskeletal structure unique to the sperm flagellar principal piece. Evidence is accumulating that the fibrous sheath serves as a scaffold for signal transduction and glycolysis in the distal flagellum, where CABYR plays a role in calcium signaling.

CABYR has recently been identified as being a cancer-testis (CT) antigen. CT antigens are a class of tumor antigens with expression restricted to male germ cells in the testis and various types of cancer, with expression typically not found in adult somatic tissues. Since the first CT antigen MAGE-1 (subsequently renamed MAGE-A1) was reported in the early 1990s, more than 47 CT genes or gene families have been identified, and their expression has been studied in numerous cancer types to date. CT antigens are ideal targets for cancer vaccines, considering that a number of them can elicit spontaneous cellular and/or humoral immune responses in some cancer patients, that the testis is an immune privileged site, and considering the lack of HLA class I expression on the surface of germ cells. However, the expression frequency of a certain CT antigens varies with different tumor types, and many CT antigens present heterogeneous expression in the same tumor tissue. Polyvalent cancer vaccines containing epitopes of several CT antigens may provide a way to increase the effectiveness of such compositions as diagnostic tools and overcome the heterogeneity associated with some cancers. Thus, it is of great value to identify and characterize new CT antigens, particularly those that are immunogenic in human.

CABYR is a calcium-binding tyrosine phosphorylation-regulated protein isolated from human spermatozoa. According to National Center for Biotechnology Information Reference Sequence, there are six transcript variants of CABYR encoding five protein isoforms: CABYR-a, CABYR-b, CABYR-c, CABYR-d, and CABYR-e (two of the transcript variants encode the same isoform, CABYR-c). Although CABYR was initially reported to be testis specific, recently, expression of CABYR-c and CABYR-d has been observed in normal brain and especially in brain tumors using reverse transcription-PCR (RT-PCR; Hsu et al. Biochem Biophys Res Commun 2005;329:1108-17).

Luo and colleagues have also reported CABYR in lung cancers, particularly adenocarcinomas and squamous cell carcinomas (Luo et al., 2007, Clin. Cancer Res., 13:4:1288-1297). The incidence of CABYR mRNAs and CABYR protein in lung cancers was approximately 40%. One CABYR variant (281) in particular, was identified in 19/20 brain tumor samples. Of note in this study was the absence of CABYR-498 in any brain tumor sample studied. The −498 variant is the largest calcium binding isoform of CABYR. Together, the two studies that address the correlation of CABYR expression in tumors showed both the presence of CABYR mRNA and CABYR proteins. Furthermore, the studies conducted by Hsu et al, detected only a subset of splice variants being expressed in brain tumors. These observations confirm the translation of CABYR mRNAs in tumors. CABYR and other sperm proteins hypothesized to be cancer-testis antigens are further discussed in Chiriva-Internati et al. (Cancer Immunity, 2008, 8:8) and in Almeida et al. (Nucleic Acids Res. 2008, 1-4, epublished Oct. 16, 2008, doi: 10.1093/nar/gkn673)

The CABYR protein is encoded by two coding regions designated “coding region A” and “coding region B” in both human and mouse. In mouse 4 splice variants have been discovered, while in humans, 6 splice variants of CABYR are designated: CABYR- 221 (contains both CR-A & B), CABYR-281 (contains both CR-A & B), CABYR-379 (contains only CR-A), CABYR-475 (contains only CR-A), and CABYR-493 (contains only CR-A), see FIG. 2 for details. Applicants have created antisera to recombinant CABYR-A and CABYR-B proteins that allowed them to discern distinct populations of CABYR protein isoforms in the human sperm encoded by either CR-A alone and/or CR-B as well as a common population containing proteins encoded by both coding regions. Both groups of isoforms localized to the fibrous sheath cytoskeletal element. Only the recombinant CABYR-A and not the CABYR-B bound calcium in vitro, which is consistent with the hypothesis that CABYR-A is the only form that binds calcium in sperm. These observations confirmed that, despite the presence of the stop codon in CR-A, splice variants containing CR-B are translated during spermiogenesis and assemble into the fibrous sheath of the principal piece; however, calcium binding occurs only to those CABYR isoforms containing CABYR-A.

The proline rich extension domain in CABYR-281 and -379 has been shown to include a binding site for glycogen synthetase kinase 3β. Further, GSK3β can phosphorylate CABYR-379 in vitro. Thr¹⁵¹-Pro and Ser¹¹⁵-Pro sites are essential for in vitro phosphorylation to occur. CABYR phosphopeptides have been identified and mapped by ms/ms during in vitro capacitation of human sperm. Seven serine/threonine sites that are phosphorylated during capacitation have been identified.

The acidic isoforms of CABYR bind calcium. The N terminus of CABYR contains a RII dimerization domain. This domain is homologous to the RII region of protein kinase A and is thought to mediate CABYR binding to A Kinase Anchoring Proteins [AKAP3 and AKAP4] that lie in the core of the sperm's fibrous sheath.

In order to gain further insight into proteins with which CABYR interacts, partner proteins present in CABYR containing immuno-precipitates have been studied by ms/ms. A novel high molecular weight phosphoprotein, FSCB (Fibrous Sheath CABYR Binding), has been identified as a CABYR partner and shown to localize to the surface of the ribs and columns of the fibrous sheath. CABYR immune complexes also contained the known kinase, JAK1, JNK, MAP kinase scaffold protein 3, ropporin, enolase, and the Unc-51 like kinase. One or more components of this complex may serve as CABYR adaptor proteins.

Squamous cell carcinomas represent by far the predominant form of cervical cancer with the majority due to HPV. The Papanicolaou smear and HPV testing for cervical precancer are the most important screening tools today. There are approximately 11,000 new cases of cervical cancer in the US annually with about 3,700 deaths. Many squamous cell carcinomas of the cervix present at low stage and are treated effectively with radiation therapy, therefore only small biopsies are usually available. HPV is also likely responsible for a proportion of head & neck and lung cancers, although the majority are due to tobacco use. In 2002, there were 170,000 new cases of cancer (all types) of the lung with 155,000 deaths annually. Approximately 40% of lung carcinomas are squamous cell carcinomas, and the vast majority are due to tobacco smoking. Squamous cancers of the lung when presenting at low stage are treated with surgery, while therapy for high stage tumors is typically ineffective. There are no serum biomarkers in routine clinical usage for screening of squamous lung cancers or for detecting recurrence.

Head and Neck Squamous Cell Carcinomas provide the best opportunity to study squamous carcinomas. The term “head and neck squamous cell carcinoma” (HNSCC) generally refers to squamous cell carcinomas (SCC) of the upper aerodigestive tract, most commonly involving the oral cavity, pharynx, and larynx. The American Cancer Society estimates that, in the U.S. in 2007, there will be 45,660 new cases of HNSCC, with 11,210 deaths. This will represent 3.2% of all new cancers and 2.0% of all cancer deaths. For patients with oral and pharyngeal SCC presenting with isolated local disease, regional metastasis, or distant metastasis, the five-year survival rates are 81%, 52%, and 26%, respectively. The overall five-year survival rate is 60%, only a slight improvement from 53% in the late 1970s. HNSCC is the sixth most frequent cancer worldwide and represents almost 50% of all malignancies in some developing nations. In many of these countries, advanced treatment approaches are not readily available.

These statistics highlight the need for a better understanding of HNSCC at the cellular and molecular levels. Identification of specific molecular defects in these cancers is guiding development of targeted molecular therapies (TMTs) which are sought after aggressively because of their potentially high success rate with reduced toxicity. Since cancer involves dysregulation of growth control systems, significant effort has been invested into understanding normal and abnormal function of growth factors in order to identify and exploit new therapeutic targets.

The EGF receptor (“EGFR”) is a transmembrane receptor that binds epidermal growth factor (“EGF”), transforming growth factor-alpha (TGF-α), and other ligands. It is a member of the HER/ErbB family. Its intracellular domain contains intrinsic tyrosine kinase (TK) activity. Like other members of the receptor TK (RTK) family, transphosphorylation of the kinase domain on tyrosine residues is required for activation, and results from ligand binding and subsequent receptor dimerization. Upon activation, phosphorylated tyrosine residues interact with various effectors, activating of the Ras/MEK/Erk and PI3K/Akt/mTOR pathways.

The EGFR regulates cell growth, differentiation, survival, metabolism and migration. Altered EGFR function is associated with oncogenic transformation, autonomous cell growth, invasion, angiogenesis, and metastasis. More than 90% of HNSCCs overexpress the EGFR and elevated EGFR expression is a predictor of decreased survival for HNSCC patients. Activated EGFR has been detected in up to 90% of specimens, and constitutive activation of downstream EGFR targets, including Erk and Akt, has also been observed. Overexpression of other HER family members has been noted in HNSCC, although much less commonly. In addition, activating mutations of the EGFR have been described. The most common, observed in 42% of HNSCC specimens studied, is EGFRvIII, a truncated 150-kDa protein that is weakly constitutively phosphorylated in the absence of ligand. The presence of mRNA for various EGFR ligands has been demonstrated in HNSCC cell lines, including EGF, HB EGF, TGF-α, amphiregulin, betacellulin, and the heregulins, implying the presence of an autocrine loop.

The level of TGF-α is a statistically significant negative predictor of disease-free survival. A variety of preclinical models showed sensitivity to EGFR inhibition by several methods, including small molecule tyrosine kinase inhibitors (TKIs), anti-EGFR monoclonal antibodies (mAbs), antisense oligonucleotides, and immunotoxin conjugates. These types of targeted agents are particularly desirable because they would be expected to have limited toxicity when compared to traditional cytotoxic agents. Unfortunately, targeted EGFR antagonists have had only modest success in the clinical treatment of HNSCC.

Several targeted anti-EGFR agents have been developed. The EGFR TKIs include gefitinib (Iressa), erlotinib (Tarceva), and lapatinib (Tykerb); they selectively bind to and inhibit the intracellular TK domain of EGFR. They are taken orally on a daily basis and adverse reactions are relatively modest, including rash, diarrhea, and nausea. The anti-EGFR mAbs include cetuximab (Erbitux) and panitumumab (Vectibix). Both are directed against the extracellular ligand binding domain of the EGFR. They are administered intravenously every 1-3 weeks. Adverse effects include rash and hypersensitivity reactions. These agents have shown limited antitumor effects when used as single agents. A phase II clinical trial of gefitinib showed an 11% response rate, with mean time to progression and mean overall survival of 3.4 and 8.1 months, respectively. A similar study with erlotinib (Tarceva) showed a 5% response rate. The net benefit of a targeted agent in combination therapy with a standard cytotoxic agent was similar. When cetuximab was combined with cisplatin for metastatic or recurrent HNSCC, there was a 26% response rate compared to 10% for cisplatin alone. Other studies have shown similar benefits of combined drug therapy. When combined with RT, cetuximab improved locoregional control and survival compared to RT alone. Based on tissue analysis from treated patients, treatment failures do not appear to result from lack of EGFR inhibition in vivo. While level of EGFR expression does not predict effectiveness of these agents, development of cutaneous toxicity is associated with greater response and positive outcome. To date, reliable molecular markers that predict response or resistance have not been elucidated for SCC.

Based on the above considerations it is becoming clear that the efficacy of targeted anti-EGFR therapy in HNSCC is limited by both intrinsic and acquired resistance, as exhibited by the low initial response rate and the limited duration of benefit, respectively. The molecular mechanism(s) of resistance likely involve redundant and/or compensatory signaling through crucial growth and/or survival pathways that are maintained or established when EGFR signaling is lost due to the introduction of an EGFR antagonist. It has been demonstrated that the response to gefitinib and cetuximab in lung cancer cells correlates with suppression of the downstream effectors Erk and Akt, and that therapeutic resistance is associated with failure to suppress the activity of these molecules. The most likely candidates to activate these molecules in a EGFR- independent fashion include (1) other growth factor or cytokine receptors that signal via similar downstream pathways, or (2) altered expression or function of a downstream element of the “resistance pathway.” Thus, it seems likely that successful targeted therapy will ultimately require a combination of inhibitors.

There is a long felt need in the art for biomarkers of cancer, particularly squamous cell carcinomas of the head and neck. The present invention satisfies these needs.

### SUMMARY OF THE INVENTION

The present invention is based on the discovery that proteins identified as testis or sperm-specific antigens can be used as biomarkers for cancer, i.e., outside the testis and sperm, only cancer cells express the biomarker(s). One such antigen includes the protein CABYR (Naaby-Hansen et al., 2002, Dev. Biol., 242:236-254).

Experiments described herein indicate a minimum of 14% of various types of squamous head & neck and lung tumors express CABYR at levels readily detected by immunohistochemistry. Accordingly, in one embodiment a method of screening for squamous cell cancer in a patient is provided and more particularly a method for detecting squamous cell head & neck and lung tumors is provided. The method comprises detecting the expression of the sperm flagellar protein, CABYR, in a biological sample of said subject. The biological sample may be a tissue sample recovered from a biopsy or a biological fluid recovered from the patient including for example, blood or sera. Detection of CABYR can be based on the identification of CABYR RNA transcripts or by detection of CABYR amino acid sequences, using standard techniques known to those skilled in the art. In one embodiment the biological tissue is screened for expression of specific variants of CABYR. In one embodiment the biological sample is serum wherein either the amount of CABYR transcripts or CABYR antigen (or the specific variant type) is diagnostic for the presence of squamous cell head & neck and lung tumors in the patient.

The present invention also encompasses opportunities for personalized medicine. The future of personalized tumor therapy is envisioned to couple sensitive diagnostic methods, which characterize a tumor's phenotype of upregulated drugable targets and pathways, to selective small molecule antagonists against targets specific to a given patient.

One hypothesis to explain the appearance of CABYR or other so-called sperm or testis-specific proteins in cancers posits that when certain tumors dysregulate they revert to the gametogenic pathway. This hypothesis implies that other post-meiotic genes unique to spermiogenesis may also appear in certain tumors. Because genes such as CABYR are normally restricted to the gonad, where they are only found in post-meiotic cells, they may be useful cancer drug targets with potential for exquisite selectivity. Drugs directed to these targets would spare the testis's regenerative capacity (because the target does not appear in spermatogonial stem cells) and would be selective to those tumors in which they dysregulate, because the protein is not expressed in other tissues. Because many tumor therapies currently are nonselective and address targets common to many cells, targeting a tumor specific protein would represent an important advance.

Another aspect regarding the prognostic value of CABYR as a biomarker stems from its known association with the fibrous sheath which has evolved as a scaffold for the assembly of glycolytic enzymes. An important underlying hypothesis in studying CABYR is that tumors in which CABYR is expressed may have dysregulated into an anaerobic phenotype having reverted to a greater dependency on glycolysis over oxidative metabolism.

In one aspect, an antibody directed against CABYR, or directed against another protein of interest, can be used to detect and measure CABYR. In another aspect, a cocktail (two or more) antibodies can be used to detect and measure CABYR, or distinguish between the six expressed variants when the antibodies are labeled with separately detectable markers.

In one aspect, the proteins and peptides of the invention, as well as fragments, homologs, derivatives, mutants, and splice variants thereof, are useful biomarkers for cancer. In one aspect, the cancers include, but are not limited to, squamous cell carcinoma. In one aspect, the squamous cell carcinoma is selected from the group consisting of head and neck, lung, and cervix. In one aspect, the cancer is brain cancer. In another aspect, the cancer is lung cancer. The etiology of the cancer is not important, as long as a protein or peptide of the invention can be used as a biomarker to detect the cancer. In one aspect, the biomarker is useful for not only detecting cancer, but also for monitoring the progression of tumor growth, as well as monitoring progression of treatment of the cancer.

One of ordinary skill in the art will appreciate that the biomarkers of the invention can also be used in conjunction with other biomarkers useful for diagnosing cancer and for monitoring the progression of cancer. It will also be appreciated that various tissues and cells can be used as samples. These include, but are not limited to, biopsies, blood, plasma, serum, urine, sputum, and feces. The results obtained using these samples can be compared to known standards, with samples from subjects without cancer, or with samples obtained from the subject of interest at various times during the progression of the cancer or treatment of cancer.

Various assays can be used to detect and measure the biomarkers of the invention. These assays include, but are not limited to, immunofluorescence, immunoblots, other immunoassays, western blots, northern blots, and spectroscopy techniques such as Ms/Ms. In some cases, a functional assay can be used.

In one aspect, a biomarker can be associated with the stage of differentiation of the cells of the cancer.

In one aspect, the biomarkers of the invention are also targets for cancer therapy. Because genes such as CABYR are normally restricted to the gonad, where they are only found in post-meiotic cells, they may be useful cancer drug targets. Drugs directed to these targets would spare the testis's regenerative capacity because the target does not appear in spermatogonial stem cells and would be selective to those tumors in which they dysregulate. In one aspect, the biomarkers of the invention may be useful as vaccines.

The biomarkers of the invention are also useful for retrospective study of tumors. For example, using samples already in storage, one can identify those that are CABYR positive. The incidence of expression of the biomarkers of the invention can also be determined retroactively. These results can also be correlated with patient history and outcome. The retrospective analysis is also useful for determining the prognostic value CABYR has in predicting tumor progression, metastasis, resection, after drug and radiation treatment, etc.

Various aspects and embodiments of the invention are described in further detail below.

### BRIEF DESCRIPTION OF THE DRAWINGS

FIG. 1. Monoclonal antibodies H4 (culture supernatant), A4 and A5 (protein A purified IgGs) are shown immunoreacting with both recombinant human CABYR (rec) as well as the native CABYR molecule isolated from human sperm (h.sp.). Results with these three monoclonal Abs are compared with previously published immune (Imm) and pre-immune (Pre-imm) polyclonal antibodies to human CABYR (right panel, positive control).

FIG. 2 is a schematic drawing of the functional domains of CABYR and demonstrating the alternative spliced variants of CABYR.

FIG. 3 presents data form an RT PCR analysis for expression of CABYR transcripts in 16 human lung tumor (T) and adjacent non-tumor (N) tissues.

FIG. 4 is a schematic drawing of the functional domains in CABYR isoform III

FIG. 5A-5C presents data demonstrating CABYR mRNA expression in lung cancer cell lines H226 and A549 relative testicular tissue (T).

### DETAILED DESCRIPTION

Definitions

In describing and claiming the invention, the following terminology will be used in accordance with the definitions set forth below. Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein. As used herein, each of the following terms has the meaning associated with it in this section. Specific and preferred values listed below for radicals, substituents, and ranges are for illustration only; they do not exclude other defined values or other values within defined ranges for the radicals and substituents.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As use herein, the terms “administration of” and or “administering” a compound should be understood to mean providing a compound of the invention or a prodrug of a compound of the invention to a subject in need of treatment.

The term “adult” as used herein, is meant to refer to any non-embryonic or non-juvenile subject. For example the term “adult adipose tissue stem cell,” refers to an adipose stem cell, other than that obtained from an embryo or juvenile subject.

Cells or tissue are “affected” by an injury, disease or disorder if the cells or tissue have an altered phenotype relative to the same cells or tissue in a subject not afflicted with the injury, disease, condition, or disorder.

As used herein, an “agonist” is a composition of matter that, when administered to a mammal such as a human, enhances or extends a biological activity of interest. Such effect may be direct or indirect.

A disease, condition, or disorder is “alleviated” if the severity of a symptom of the disease or disorder, the frequency with which such a symptom is experienced by a patient, or both, are reduced.

As used herein, “alleviating an injury, disease or disorder symptom,” means reducing the frequency or severity of the symptom.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

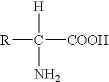

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino-and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

An “antagonist” is a composition of matter that when administered to a mammal such as a human, inhibits or impedes a biological activity attributable to the level or presence of an endogenous compound in the mammal. Such effect may be direct or indirect.

The term “antimicrobial agents” as used herein refers to any naturally-occurring, synthetic, or semi-synthetic compound or composition or mixture thereof, which is safe for human or animal use as practiced in the methods of this invention, and is effective in killing or substantially inhibiting the growth of microbes. “Antimicrobial” as used herein, includes antibacterial, antifungal, and antiviral agents.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

A ligand or a receptor (e.g., an antibody) “specifically binds to” or “is specifically immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988, Antibodies, A Laboratory Manual, Cold Spring Harbor Publications, New York) for a description of immunoassay formats and conditions that can be used to determine specific immunoreactivity.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

The term “biological sample,” as used herein, refers to samples obtained from a living organism, including skin, hair, tissue, blood, plasma, cells, sweat, and urine.

As used herein, the term “biologically active fragments” or “bioactive fragment” of the polypeptides encompasses natural or synthetic portions of the full-length protein that are capable of specific binding to their natural ligand or of performing the function of the protein.

A “biomarker” is a specific biochemical in the body which has a particular molecular feature that makes it useful for measuring the progress of disease or the effects of treatment, or for measuring a process of interest.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize), as well as any of a number of characteristic structural and/or molecular features. A “cancerous” or “malignant cell” is understood as a cell having specific structural properties, lacking differentiation and being capable of invasion and metastasis. Examples of cancers are, breast, lung, brain, bone, liver, kidney, colon, and prostate cancer. (see DeVita, V. et al. (eds.), 2001, Cancer Principles and Practice of Oncology, 6th. Ed., Lippincott Williams & Wilkins, Philadelphia, Pa.; this reference is herein incorporated by reference in its entirety for all purposes).

“Cancer-associated” refers to the relationship of a nucleic acid and its expression, or lack thereof, or a protein and its level or activity, or lack thereof, to the onset of malignancy in a subject cell. For example, cancer can be associated with expression of a particular gene that is not expressed, or is expressed at a lower level, in a normal healthy cell. Conversely, a cancer-associated gene can be one that is not expressed in a malignant cell (or in a cell undergoing transformation), or is expressed at a lower level in the malignant cell than it is expressed in a normal healthy cell.

As used herein, the term “carrier molecule” refers to any molecule that is chemically conjugated to the antigen of interest that enables an immune response resulting in antibodies specific to the native antigen.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates or carboxylic acids, on the antigen and the carrier molecule. Chemical conjugation also includes non-covalent linkage between the antigen and the carrier molecule.

The term “competitive sequence” refers to a peptide or a modification, fragment, derivative, or homolog thereof that competes with another peptide for its cognate binding site.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.”

Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “complex”, as used herein in reference to proteins, refers to binding or interaction of two or more proteins. Complex formation or interaction can include such things as binding, changes in tertiary structure, and modification of one protein by another, such as phosphorylation.

A “compound,” as used herein, refers to any type of substance or agent that is commonly considered a drug, or a candidate for use as a drug, as well as combinations and mixtures of the above.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “diagnosis” refers to detecting cancer or a risk or propensity for development of cancer, for the types of cancer encompassed by the invention. In any method of diagnosis there exist false positives and false negatives. Any one method of diagnosis does not provide 100% accuracy.

A “disease” is a state of health of an animal wherein the subject cannot maintain homeostasis, and wherein if the disease is not ameliorated then the subject's health continues to deteriorate. In contrast, a “disorder” in an subject is a state of health in which the animal is able to maintain homeostasis, but in which the subject's state of health is less favorable than it would be in the absence of the disorder. Left untreated, a disorder does not necessarily cause a further decrease in the subject's state of health.

As used herein, an “effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly 5 amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” molecule is a molecule in a form in which it exhibits a property or activity by which it is characterized. A functional enzyme, for example, is one that exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

The term “inhibit,” as used herein, refers to the ability of a compound, agent, or method to reduce or impede a described function, level, activity, rate, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block.”

As used herein “injecting or applying” includes administration of a compound of the invention by any number of routes and means including, but not limited to, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein the term “expression” when used in reference to a gene or protein, without further modification, is intended to encompass transcription of a gene and/or translation of the transcript into a protein.

“Malexpression” of a gene means expression of a gene in a cell of a patient afflicted with a disease or disorder, wherein the level of expression (including non-expression), the portion of the gene expressed, or the timing of the expression of the gene with regard to the cell cycle, differs from expression of the same gene in a cell of a patient not afflicted with the disease or disorder. It is understood that malexpression may cause or contribute to the disease or disorder, be a symptom of the disease or disorder, or both.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

The term “material” refers to any compound, molecule, substance, or group or combination thereof that forms any type of structure or group of structures during or after electroprocessing. Materials include natural materials, synthetic materials, or combinations thereof. Naturally occurring organic materials include any substances naturally found in the body of plants or other organisms, regardless of whether those materials have or can be produced or altered synthetically. Synthetic materials include any materials prepared through any method of artificial synthesis, processing, or manufacture. Preferably, the materials are biologically compatible materials.

As used herein, the terms “native”, “natural” “native antigen”, or “natural antigen” refers to the antigen as it occurs in nature. With respect to the invention, the “native antigens” are of “low immunogenicity.” “Low immunogenicity” refers to the inability of the natural molecule to elicit a strong immune response resulting in the production of high affinity antibodies. The term “antigen”, “antigen of interest,” or specific molecules, such as the cancer-testis antigens encompassed herein include the whole molecule or any portions thereof that maintain antigenic distinctiveness specific for the native antigen.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

The term “Oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Operably linked” refers to a juxtaposition wherein the components are configured so as to perform their usual function. Thus, control sequences or promoters operably linked to a coding sequence are capable of effecting the expression of the coding sequence. By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

“Permeation enhancement” and “permeation enhancers” as used herein relate to the process and added materials which bring about an increase in the permeability of skin to a poorly skin permeating pharmacologically active agent, i.e., so as to increase the rate at which the drug permeates through the skin and enters the bloodstream. “Permeation enhancer” is used interchangeably with “penetration enhancer”.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

By “presensitization” is meant pre-administration of at least one innate immune system stimulator prior to challenge with a pathogenic agent. This is sometimes referred to as induction of tolerance.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “prevent,” as used herein, means to stop something from happening, or taking advance measures against something possible or probable from happening. In the context of medicine, “prevention” generally refers to action taken to decrease the chance of getting a disease or condition.

A “prophylactic” treatment is a treatment administered to a subject who does not exhibit signs of a disease or injury or exhibits only early signs of the disease or injury for the purpose of decreasing the risk of developing pathology associated with the disease or injury.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA. A “significant detectable level” is an amount of contaminate that would be visible in the presented data and would need to be addressed/explained during analysis of the forensic evidence.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates. The terms “protein pathway” and “protein regulatory pathway” are used interchangeably herein.

The term “regulate” refers to either stimulating or inhibiting a function or activity of interest.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

As used herein, the term “secondary antibody” refers to an antibody that binds to the constant region of another antibody (the primary antibody).

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

“Sperm-specific”, as used herein, refers to an antigen which is present at higher levels on sperm than other cells, or is exclusively present in sperm.

A “test sample”, as used herein, refers to a sample of semen or to a sample obtained as a forensic sample such as a post-coital swab.

Used interchangeably herein are the following pairs of words (1) “detect” and “identify”; (2) “select” and “isolate”; and (3) “sperm surface” and “sperm plasma membrane.”

The term “standard,” as used herein, refers to something used for comparison. For example, a standard can be a known standard agent or compound which is administered or added to a control sample and used for comparing results when measuring said compound in a test sample. In one aspect, the standard compound is added or prepared at an amount or concentration that is equivalent to a normal value for that compound in a normal subject. Standard can also refer to an “internal standard,” such as an agent or compound which is added at known amounts to a sample and is useful in determining such things as purification or recovery rates when a sample is processed or subjected to purification or extraction procedures before a marker of interest is measured.

A “subject” of analysis, diagnosis, or treatment is an animal. Such animals include mammals, preferably a human.

As used herein, a “subject in need thereof” is a patient, animal, mammal, or human, who will benefit from the method of this invention.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

“Tissue” means (1) a group of similar cells united to perform a specific function; (2) a part of an organism consisting of an aggregate of cells having a similar structure and function; or (3) a grouping of cells that are similarly characterized by their structure and function, such as muscle or nerve tissue.

The term to “treat,” as used herein, means reducing the frequency with which symptoms are experienced by a patient or subject or administering an agent or compound to reduce the frequency with which symptoms are experienced.

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

“Treating” is used interchangeably with “treatment” herein.

By the term “vaccine,” as used herein, is meant a composition which when inoculated into an animal has the effect of stimulating an immune response in the subject, which serves to fully or partially protect the subject against a disease or its symptoms. The term vaccine encompasses prophylactic as well as therapeutic vaccines. A combination vaccine is one which combines two or more vaccines, or two or more compounds or agents.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### Embodiments

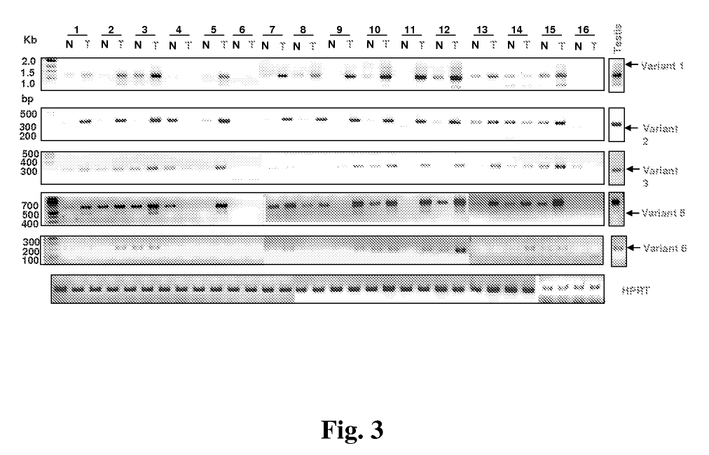

The calcium binding tyrosine phosphorylated protein (CABYR) has been previously characterized as a testis-specific calcium-binding protein. In humans CABYR is expressed as six different variants (see FIG. 3). Applicants have discovered that several of the six variants are expressed in squamous cells of head and neck and lung tumors. Accordingly, as disclosed herein CABYR can serve as a diagnostic marker for cancers, including for example, squamous cell cancers.

In accordance with one embodiment a method is provided for screening patients for the presence of tumor cells. More particularly, the method is suited for diagnosing patients having a squamous cell cancer, including a head and neck squamous cell tumor or lung cancer. The method is based on the detection of CABYR expression in non-testicular tissues and cells. Detection of either CABYR mRNA transcripts and/or CABYR peptide sequences in tissues other than testicular tissues and cells has been correlated with neoplastic cells. Therefore, in accordance with one embodiment a method for screen individuals for the presence of tumors is provided. The method comprises obtaining a biological sample from the patient and subjecting the sample to analysis to determine if the sample comprises CABYR mRNA or peptide sequences.

In one embodiment, the biological sample will be analyzed for the presence of RNA transcripts of the CABYR gene. More particularly, the sample will be analyzed to determine if RNA transcripts are present that have sequences identical to SEQ ID NO: 1, SEQ ID NO: 3, SEQ ID NO: 5, SEQ ID NO: 7, SEQ ID NO: 9 or SEQ ID NO: 11. In one embodiment the sample will be analyzed to determine if RNA transcripts are present that have sequences identical to SEQ ID NO: 1, SEQ ID NO: 3, SEQ ID NO: 5, SEQ ID NO: 9 or SEQ ID NO: 11. In one embodiment, the sample will be analyzed to determine if RNA transcripts are present that have sequences identical to SEQ ID NO: 5. In one embodiment, the biological sample will be analyzed using standard techniques, including for example, RT-PCR and Northern and Southern blot analysis, to detect RNA's that comprise a 10 nucleotide or longer sequence that is identical to a contiguous sequence selected from the group consisting of SEQ ID NO: 1, SEQ ID NO: 3, SEQ ID NO: 5, SEQ ID NO: 7, SEQ ID NO: 9 or SEQ ID NO: 11. In one embodiment the biological sample will be analyzed to detect RNA's that comprise a 10 nucleotide or longer sequence that is identical to a contiguous sequence of SEQ ID NO: 5 (encoding CABYR variant 3). In one embodiment the sample will be screened for RNA transcripts that encodes a peptide comprising at least 6 amino acids identical to a contiguous sequence of SEQ ID NO: 6.

In an alternative embodiment the biological sample will analyzed to detect peptide sequences of the sperm flagellar protein, CABYR. In one embodiment the sample is screened for the presence of a peptide sequence comprising at least 6 amino acids identical a sequence selected from the group consisting of SEQ ID NO: 2, SEQ ID NO: 4, SEQ ID NO: 6, SEQ ID NO: 8, SEQ ID NO: 10 and SEQ ID NO: 12. In one embodiment the sample is screened for the presence of a peptide sequence comprising at least 6 amino acids identical a sequence selected from the group consisting of SEQ ID NO: 2, SEQ ID NO: 4, SEQ ID NO: 6, SEQ ID NO: 10 and SEQ ID NO: 12. In one embodiment the sample is screened for the presence of a peptide sequence comprising at least 6 amino acids identical to the sequence of SEQ ID NO: 6. In one embodiment the expression of CABYR peptides is detected by an immunoassay using an antibody that specifically binds to a peptide selected from the group consisting of SEQ ID NO: 2, SEQ ID NO: 4, SEQ ID NO: 6, SEQ ID NO: 8, SEQ ID NO: 10 and SEQ ID NO: 12. In one embodiment the immunoassay uses an antibody that specifically binds to a peptide selected from the group consisting of SEQ ID NO: 6, SEQ ID NO: 10 and SEQ ID NO: 12, and in one embodiment the antibody specifically binds to a peptide of SEQ ID NO: 6 without cross reacting with the other CABYR variant proteins.

The methods of detecting and measuring cancer biomarkers using sperm-specific antibodies can employ a variety of detectable markers, or reporter molecules, that are either directly linked or indirectly linked to the sperm-specific antibodies. Such detectable markers or reporter molecules include, but are not limited to, colorimetric molecules, fluorescent molecules, chemiluminescent molecules, or horseradish peroxidase (HRP).

Under suitable conditions, a colorimetric reporter molecule forms a color or changes color, a fluorescent reporter molecule fluoresces or changes fluorescence, and a chemiluminescent reporter molecule chemiluminesces, or emits light due to a chemical reaction. Horseradish peroxidase (HRP) may be considered to be a colorimetric reporter molecule. An antibody-HRP conjugate causes precipitation of a colored substrate where the antibody binds to the corresponding antigen.

A reporter molecule may be an enzyme or an enzyme substrate. If the reporter molecule is an enzyme, the corresponding enzyme substrate is added after the antibody is allowed to bind to the corresponding antigen. If the reporter molecule is an enzyme substrate, the corresponding enzyme is added. Reaction between the enzyme and the enzyme substrate gives rise to the formation of a color, a change in color, fluorescence, a change in fluorescence, or chemiluminescence.

In one embodiment, the antibodies are labeled either directly or indirectly, using an immunofluorescence compound and techniques known to those skilled in the art. In the direct method, the antibodies are labeled directly with a fluorochrome. In the indirect method, the fluorochrome is attached to a secondary antibody that recognizes the CABYR-specific antibody. In one embodiment, the CABYR-specific antibodies are monoclonal antibodies that have been directly conjugated to a fluorochrome.

The indirect method has the advantage that it can amplify the fluorescent signal by binding more fluorochrome at the antigen site. The direct method has the advantage that it reduces the number of washing steps and is quicker. The use of a single labeled immunoreagent also reduces the background fluorescence by eliminating non-specific binding of the secondary antibody. One possible drawback of using a single labeled immunoreagent is that at low antibody-antigen ratios, the fluorescent signal may be lower than that in the indirect method.

One aspect of the present invention is directed to the use of horseradish peroxidase (HRP) conjugates of CABYR-specific antibodies to immunostain samples. HRP conjugates stain cells by causing precipitation of a colored substrate where the antibody is bound to the cell. Other commercially available reporter molecules or substrates include, e.g., True Blue® (tetramethyl benzidine, TMB) from KPL Laboratories and NovaRED® from Vector Laboratories.

CABYR-specific antibodies can be bound to solid support using techniques known to those skilled in the art. For example, the antibodies can be directly linked to functional groups at the surface of the solid support or can be attached to the solid support via a linker moiety. The linkage is preferably a covalent bond, although other linkages are also acceptable. In one embodiment, the CABYR-specific antibodies are linked to the solid support via an antibody linker, where the linker is a secondary antibody that binds to the constant region of the CABYR-specific primary antibody. In another embodiment, the linker is an enzymatically cleavable or photolytic linker. Linkers suitable for use in accordance with the present invention are well known to those skilled in the art.

In one embodiment, the solid support comprises a single solid surface. In another embodiment, the solid support is in particulate form. The particles may vary in shape and can be, e.g., round, rectangular, or irregularly shaped. Irregular shape adds more surface area, increasing the particles' binding capacity compared to larger spherical particles. The particles may also vary in size. Smaller particles may be more diffused throughout the sample solution, increasing target capture rate while decreasing incubation time. Preferably, the size of the particles is less than 4 μm, more preferably from about 10 nm to about 1 μm, and even more preferably from about 50 nm to about 500 nm. In a particular embodiment, the size of the particles ranges from about 100 nm to about 300 nm. In one embodiment, the solid support particles, to which are bound CABYR-specific antibodies, are combined to form a column, and the biological or forensic sample is run through the column, followed by repeated washings.

In one aspect of the invention, the solid support comprises magnetic beads or particles linked to CABYR-specific antibodies. In a particular embodiment, the antibodies are monoclonal antibodies. In one embodiment, the magnetic beads or particles are each coated with one or more different antibodies specific for different CABYR-specific antigens. In another embodiment, a mixture of different types of magnetic beads or particles is used, each bead type being coated with a different antibody specific for a different CABYR-specific antigen. The use of a bead with more than one type of antibody directed against different CABYR-specific antigens, or a mixture of different bead types coated with different antibodies directed against different sperm-specific antigens is anticipated to result in binding to a higher proportion of sperm.

A biological sample is added to a preparation including one or more antibodies specific for one or more different sperm-specific antigens. The antibodies may be polyclonal or monoclonal, preferably monoclonal. The presence of CABYR in the sample gives rise to binding between the particular antibody and CABYR for which the antibody is specific, a binding reaction which is detected, directly or indirectly, through a variety of methodologies, e.g., those described in U.S. Pat. No. 5,605,803, which is incorporated by reference herein in its entirety.

A familiar type of assay is a colorimetric assay, in which a CABYR-specific antibody is labeled with a reporter molecule which can be detected by a specific color. A binding reaction between the antibody and the corresponding CABYR-specific antigen induces the formation of a color or a color change, or the color is developed with a second agent, typically an enzyme. As the reporter molecule is “developed” (i.e., the appropriate color is induced), only in the presence of CABYR-bound antibody, a “positive” reaction is indicative of the presence of tumor cells. The absence of the desired color (or the presence of a different color) is indicative of a “negative” result, i.e., an absence of the tumor-specific antigen (CABYR).

Among the easiest assays of this type to perform are solid-phase immunoassays, in which a first CABYR-specific antibody, preferably a monoclonal antibody, is bound to a solid surface, such as a membrane or bed, which is exposed to a sample. Any biomarker present in the sample binds to the first antibody. Any unbound or non-specifically bound material is washed or removed from the solid surface, followed by the addition of a second antibody which binds to a CABYR-specific antigen and bears a reporter molecule or a label such as an enzyme or enzyme substrate. The second antibody need not bind to the same CABYR epitope as the first antibody. After binding to the second antibody is allowed to occur, the solid surface is washed to remove any unbound or non-specifically bound material. Conditions are then established so that the reporter molecule or label may give a readily detectable signal which is indicative of the presence of CABYR.

If the second antibody is labeled with an enzyme or enzyme substrate, the counterpart of the enzyme or enzyme substrate is added after washing the solid surface. (When the second antibody is bound to an enzyme, the enzyme substrate is added. When the second antibody is bound to the enzyme substrate, the enzyme is added.) The enzyme cleaves a portion of the enzyme substrate, causing the substrate to form a color, to undergo a color change, to chemiluminesce, or to fluoresce, or causing some other readily detectable phenomenon. In one embodiment, the various elements of the assay, including the solid phase-bound first antibody, the labeled second antibody, and the enzyme or enzyme substrate, are furnished in a single kit used to demonstrate the presence or absence of CABYR in a biological or forensic sample.

In one embodiment, a first CABYR-specific antibody, preferably a monoclonal antibody, is contacted with a biological sample under conditions (e.g., aqueous sample, ambient temperature, and normal atmosphere) which permit the antibody-CABYR antigen binding reaction to occur. After sufficient reaction time has passed, to the preparation is added a second CABYR-specific antibody, which may or may not bind to the same epitope as the first antibody. The second antibody, bearing a reporter molecule or a label such as an enzyme or enzyme substrate, is allowed to bind to CABYR bound by the first antibody. In a particular embodiment, the label conjugated to the second antibody is an enzyme. Any unbound or non-specifically bound material, including the second antibody, is removed, e.g., by pouring or washing off the sample. In one embodiment, the first antibody is bound to a solid surface to make the assay simpler and more “user friendly.” A substrate which forms a color, changes color, chemiluminesces, fluoresces, or undergoes some other readily detectable change in the presence of the enzyme, due to the action of the enzyme on the substrate, is then added. Representative enzyme immunosorbent assays (EIA) are described in U.S. Pat. No. 5,149,622, which is incorporated by reference herein in its entirety. Other solid- and liquid-phase assay methodologies may be employed without the exercise of inventive skill.

One embodiment of the invention follows the capture assay format, in which a CABYR-specific monoclonal antibody is bound to a solid phase and used to capture the corresponding CABYR-specific antigen. Recognition of the CABYR-specific antigen may be completed by the use of a second CABYR-specific monoclonal or polyclonal immunoreagent coupled to a reporter enzyme, or a third immunoreagent may be employed in a sandwich, as described in Shen et al., 1993, Am. J. Reprod. Immunology 29:231-240.

Another type of assay utilizes a wick (dip stick) and colored beads coated w/ a first CABYR-specific antibody. A drop of a biological sample is applied to the antibody-coated colored beads and the beads bound by CABYR migrate through a wick until they are captured by a second CABYR-specific antibody, which may or may not bind to the same epitope as the first antibody.

Yet another kind of assay employs colored magnetic beads coated w/ a first CABYR-specific antibody, which may be monoclonal or polyclonal. After mixing the beads with a sample, any cells expressing CABYR are captured by the antibody-coated beads. A magnetic dipstick may be used to recover the magnetic beads. The magnetic source is then deactivated to release the colored magnetic beads. The beads are then allowed to migrate in a wick to a zone containing a second sperm-specific antibody, which captures the sperm-bound beads, resulting in a colored line.

In yet another assay format, specialized glass beads with silanized microspikes are employed.

Antibodies directed against sperm-specific polypeptides or peptide fragments thereof may be generated using methods that are well known in the art. For instance, U.S. patent application Ser. No. 07/481,491, which is incorporated by reference herein in its entirety, discloses methods of raising antibodies to sperm-specific proteins. For the production of antibodies, various host animals, including but not limited to rabbits, mice, and rats, can be immunized by injection with a sperm-specific polypeptide or peptide fragment thereof. To increase the immunological response, various adjuvants may be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For the preparation of monoclonal antibodies, any technique which provides for the production of antibody molecules by continuous cell lines in culture may be utilized. For example, the hybridoma technique originally developed by Kohler and Milstein (1975, Nature 256:495-497), the trioma technique, the human B-cell hybridoma technique (Kozbor et al., 1983, Immunology Today 4:72), and the EBV-hybridoma technique (Cole et al., 1985, in Monoclonal Antibodies and Cancer Therapy, Alan R. Liss, Inc., pp. 77-96) may be employed to produce human monoclonal antibodies. In another embodiment, monoclonal antibodies are produced in germ-free animals utilizing the technology described in international application no. PCT/US90/02545, which is incorporated by reference herein in its entirety.

In accordance with the invention, human antibodies may be used and obtained by utilizing human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, Inc., pp. 77-96). Furthermore, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for epitopes of SLLP polypeptides together with genes from a human antibody molecule of appropriate biological activity can be employed; such antibodies are within the scope of the present invention. Once specific monoclonal antibodies have been developed, the preparation of mutants and variants thereof by conventional techniques is also available.

In one embodiment, techniques described for the production of single-chain antibodies (U.S. Pat. No. 4,946,778, incorporated by reference herein in its entirety) are adapted to produce protein-specific single-chain antibodies. In another embodiment, the techniques described for the construction of Fab expression libraries (Huse et al., 1989, Science 246:1275-1281) are utilized to allow rapid and easy identification of monoclonal Fab fragments possessing the desired specificity for sperm-specific antigens, proteins, derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment; the Fab fragments which can be generated by treating the antibody molecule with papain and a reducing agent; and Fv fragments.

The generation of polyclonal antibodies is accomplished by inoculating the desired animal with the antigen and isolating antibodies which specifically bind the antigen therefrom.

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

A nucleic acid encoding the monoclonal antibody obtained using the procedures described herein may be cloned and sequenced using technology which is available in the art, and is described, for example, in Wright et al. (1992, Critical Rev. in Immunol. 12(3,4):125-168) and the references cited therein. Further, the antibody of the invention may be “humanized” using the technology described in Wright et al., (supra) and in the references cited therein, and in Gu et al. (1997, Thrombosis and Hematocyst 77(4):755-759).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al., (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol.248:97-105).

In the production of antibodies, screening for the desired antibody can be accomplished by techniques known in the art, e.g., ELISA (enzyme-linked immunosorbent assay). Antibodies generated in accordance with the present invention may include, but are not limited to, polyclonal, monoclonal, chimeric (i.e., “humanized”), and single chain (recombinant) antibodies, Fab fragments, and fragments produced by a Fab expression library.

In accordance with one embodiment an antibody capable of binding specifically to a polypeptide selected from the group consisting of SEQ ID NO: 2, SEQ ID NO: 4, SEQ ID NO: 6, SEQ ID NO: 8, SEQ ID NO: 10 and SEQ ID NO: 12 is provided. In one embodiment the antibody specifically binds to a polypeptide comprising the sequence of SEQ ID NO: 6. In one embodiment the antibody is a monoclonal antibody, a bispecific antibody, a chimeric antibody, or a humanised antibody. In a further embodiment the antibody is conjugated to a therapeutic moiety, said therapeutic moiety selected from the group consisting of a second antibody (or a fragment or derivative thereof), a cytotoxic agent and a cytokine. The antibodies disclosed herein can be combined with a carrier or diluent to form a composition. In one embodiment, the carrier is a pharmaceutically acceptable carrier. In another embodiment, the antibodies are linked to a solid support. In yet another embodiment, the antibodies are linked to a detectable marker.

In one embodiment a kit is provided comprising at least one reagent required for detecting or quantitating a CABYR nucleic acid or peptide sequence in a biological sample. The kit may further comprise additional reagents for detecting CABYR expression in a biological sample. In accordance with one embodiment the kit comprises a CABYR quantifying agent selected from the group consisting of a CABYR specific antibody, a nucleic acid sequence complementary to a CABYR gene sequence and reagents for detecting the CABYR peptide or nucleic acid sequences. In one embodiment the antibodies or nucleic acids provided with the kit are labeled, or reagents are provided for labeling the CABYR specific antibodies or nucleic acid sequences. To this end, the antibodies, nucleic acids and other reagents can be packaged in a variety of containers, e.g., vials, tubes, bottles, and the like. Other reagents can be included in separate containers and provided with the kit; e.g., positive control samples, negative control samples, buffers, etc. In one embodiment the kit is further provided with a known anti-tumor agent. The kit would also be provided with instructional materials for using the reagents to detect CABYR expression. In one embodiment the reagent for detecting CABYR expression is an antibody, and in one embodiment is an antibody specific for the peptide of SEQ ID NO: 6. In another embodiment the reagent for detecting CABYR expression is an oligonucleotide probe that comprises a complementary sequence to an CABYR mRNA, and in one embodiment the oligonucleotide probe comprises a sequence of SEQ ID NO: 5 or a complimentary sequence thereof.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the antibodies/reagents of the invention or be shipped together with a container which contains the antibody/reagents. Alternatively, the instructional material may be shipped separately from the container with the intention that the instructional material and the reagents be used cooperatively by the recipient.

The present invention also encompasses method of treating cancers, particularly patients having a squamous cell cancer, including a head and neck squamous cell tumor or lung cancer. The method comprises administering to a subject a therapeutically effective amount of at least one agent capable of modulating the expression and/or activity of the CABYR polypeptide. In accordance with one embodiment a composition can be provided comprising an agent capable of modulating the expression and/or activity of the CABYR polypeptide and an additional known anticancer agent as a means of enhancing the efficacy of the known anticancer agent. In one embodiment the agent is an antisense oligonucleotide or a siRNA olignonucleotide. In one embodiment the CABYR RNA transcripts or peptides are used to target a therapeutic agent to cancer cells. In this embodiment the therapeutic agent is linked to a ligand that will specifically bind to a CABYR RNA transcript or peptide. In one embodiment an antibody specific for a CABYR peptide is covalently linked to a therapeutic agent, and in one embodiment the antibody specifically binds to the peptide of SEQ ID NO: 6. In one embodiment the CABYR specific antibody is conjugated to a cytotoxic agent.

In accordance with another embodiment the detection of CABYR nucleic acid sequences or peptide sequences can be used as a diagnostic indicator of the effectiveness of a cancer treatment. In particular, expression of CABYR should decrease proportional to the effectiveness of the cancer treatment. Thus by detecting the expression of the sperm flagellar protein, CABYR, in a biological sample of said subject prior to initiating said treatment and continuing to monitor the expression of the sperm flagellar protein, CABYR through out the treatment, a determination can be made as to whether or not the treatment is having the desired effect and to guide optimization of the therapeutic regiment. Such monitoring can be conducting using additional cancer markers to optimize the cancer treatment of individual patients.

The biological sample obtained from the patient to be screened can be from any relevant non-testicular tissue including for example, a biopsy tissue sample or a patient bodily fluid including for example saliva, blood, sera, plasma, urine. It is known that nucleic acids from ruptured cells can be detected in the blood of humans. Accordingly, one aspect of the present invention comprises obtaining a blood sample from a subject and analyzing the sample for the presence of nucleic acid sequences that are identical to, or complimentary to, a contiguous sequence of SEQ ID NO: 1, SEQ ID NO: 3, SEQ ID NO: 5, SEQ ID NO: 7, SEQ ID NO: 9 or SEQ ID NO: 11.

In accordance with one embodiment a method is provided for screening for agents capable of interacting with at least one polypeptide of SEQ ID NO: 2, SEQ ID NO: 4, SEQ ID NO: 6, SEQ ID NO: 8, SEQ ID NO: 10 or SEQ ID NO: 12, said method comprising: (a) contacting a polypeptide of SEQ ID NO: 2, SEQ ID NO: 4, SEQ ID NO: 6, SEQ ID NO: 8, SEQ ID NO: 10 or SEQ ID NO: 12 with a candidate agent; and (b) determining if the candidate agent interacts with said polypeptide. In one embodiment the step of determining an interaction of a candidate agent with the polypeptide comprises quantitatively detecting binding of the candidate agent to said polypeptide. In one embodiment the method is used to screen for agents capable of modulating i) expression and/or activity of a CABYR polypeptide, or ii) expression of a nucleic acid molecule encoding a CABYR polypeptide. In this embodiment the method comprises: a) comparing the expression and/or activity of said polypeptide or the expression of said nucleic acid molecule in the presence of a candidate agent with the expression and/or activity of said polypeptide or the expression of said nucleic acid molecule in the absence of the candidate agent or in the presence of a control agent; and b) determining whether the presence of the candidate agent modulates the expression and/or activity of said polypeptide or the expression of the nucleic acid molecule.

The present disclosure also encompasses the preparation of compositions comprising CABYR peptide sequences for use as therapeutic cancer vaccines. It is well known that the testis is an immune privileged site due to the presence of the blood-testis barrier, and CABYR is expressed on the protected side of the blood-testis barrier. Furthermore it is also known that CABYR is immunogenic in humans. Accordingly, administration of an antigenic composition comprising a CABYR peptide, including a peptide sequence comprising at least a 6 or 10 amino acid sequence identical to a contiguous sequence of SEQ ID NO: 6, could be administered to a subject as a means of treating cancer.

The peptides of the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the a-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the a-amino of the amino acid residues, both methods of which are well known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resiniblocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl-blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high-resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C4 -, C8- or C18-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

The present invention also provides for analogs of proteins. Analogs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. To that end, 10 or more conservative amino acid changes typically have no effect on peptide function.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides or antibody fragments which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

In another embodiment disclosed herein, peptide longevity is enhanced by the addition of adducts such as sucrose or polyethylene glycol, production of peptide-IgG chimeras, or the peptides can be cyclized via cysteine-cysteine linkages, which is a modification known to enhance the biological activities of a variety of peptides.

In one aspect a polyethylene glycol adduct is (2-aminoethyl)-O′-(N-diglycolyl-2-aminoethyl)-hexaethyleneglycol. In another aspect of the invention, a polyethylene glycol adduct is in the form of GK[(2-aminoethyl)-O′-(N-diglycolyl-2-aminoethyl)-hexaethyleneglycol]GG. The dipeptide GK increases peptide solubility. The dipeptide GG is present as a spacer between the solid support and peptide chain to improve the ease of peptide synthesis.

The present disclosure also contemplates any of the peptides derivatized with functional groups and/or linked to other molecules to facilitate their delivery to specific sites of action, to potentiate their activity, or complexed covalently or non-covalently to other pharmaceuticals, bioactive agents, or other molecules. Such derivatizations must be accomplished so as to not significantly interfere with the properties of the peptides. Carriers and derivatizations must also be designed or chosen so as not to exert toxic or undesirable activities on animals or humans treated with these formulations. Functional groups which may be covalently linked to the peptides may include, but not be limited to, amines, alcohols, or ethers. Functional groups to be covalently linked to the peptides to increase their in vivo half-lives may include, but not be limited to, polyethylene glycols, small carbohydrates such as sucrose, or peptides and proteins. The peptides may also be synthesized by recombinant DNA techniques with expression vectors for use in biological systems, such as bacteria, yeast, insect, or mammalian cells.

Generally, the amount of peptide administered depends upon the degree of immune response that is desired. Those skilled in the art may derive appropriate dosages and schedules of administration to suit the specific circumstances and needs of the patient. Typically, dosages of peptide are between about 0.001 mg/kg and about 100 mg/kg body weight. In some embodiments dosages are between about 0.01 mg/kg and about 60 mg/kg body weight. In other embodiments, dosages are between about 0.05 mg/kg and about 5 mg/kg body weight.

In general, the schedule or timing of administration of a peptide of the invention is according to the accepted practice for the procedure being performed.

When used in vivo, the peptides of the invention are preferably administered as a pharmaceutical composition. The invention thus provides pharmaceutical compositions comprising a peptide, or a pharmaceutically acceptable salt thereof, and a pharmaceutically acceptable carrier. The peptide of the invention may be present in a pharmaceutical composition in an amount from 0.001 to 99.9 wt %, and more preferably from about 0.1 to 99.0 wt %. To achieve good plasma concentrations, a peptide or a combination of peptides, may be administered, for example, by intravenous injection, as a solution comprising 0.1 to 1.0% of the active agent.

The compositions of the present invention may comprise at least one active peptide, one or more acceptable carriers, and optionally other peptides or therapeutic agents.

For in vivo applications, the peptides of the present invention may comprise a pharmaceutically acceptable salt. Suitable acids which are capable of forming such salts with the compounds of the present invention include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, anthranilic acid, cinnamic acid, naphthalene sulfonic acid, sulfanilic acid and the like.

Pharmaceutically acceptable carriers include physiologically tolerable or acceptable diluents, excipients, solvents or adjuvants. The compositions are preferably sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g. 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Pharmaceutical compositions according to the present invention can be prepared in a manner fully within the skill of the art.

The peptides of the invention, pharmaceutically acceptable salts thereof, or pharmaceutical compositions comprising these compounds may be administered so that the compounds may have a physiological effect. Administration may occur enterally or parenterally; for example orally, rectally, intracisternally, intravaginally, intraperitoneally, locally (e.g., with powders, ointments or drops), or as a buccal or nasal spray or aerosol. Parenteral administration is preferred. Particularly preferred parenteral administration methods include intravascular administration (e.g. intravenous bolus injection, intravenous infusion, intra-arterial bolus injection, intra-arterial infusion and catheter instillation into the vasculature), peri- and intra-target tissue injection (e.g. peri-tumoral and intra-tumoral injection), subcutaneous injection or deposition including subcutaneous infusion (such as by osmotic pumps), intramuscular injection, and direct application to the target area, for example by a catheter or other placement device.

Where the administration of the peptide is by injection or direct application, the injection or direct application may be in a single dose or in multiple doses. Where the administration of the compound is by infusion, the infusion may be a single sustained dose over a prolonged period of time or multiple infusions.

The present application encompasses the use of siRNA for blocking the pathways identified herein. In one aspect, the siRNA is directed against CABYR or a fragment thereof. In a further aspect, a first siRNA can be used in combination with a second siRNA with a slightly different sequence than the first, or the second siRNA can be directed against a different sequence altogether. An siRNA of the invention can be further used with other regulators described herein, or known in the art, such as peptides, antisense oligonucleotides, nucleic acids encoding peptides described herein, aptamers, antibodies, kinase inhibitors, and drugs/agents/compounds.

The present disclosure also encompasses useful aptamers. In one embodiment, an aptamer is a compound that is selected in vitro to bind preferentially to another compound (in this case the identified proteins). In one aspect, aptamers are nucleic acids or peptides, because random sequences can be readily generated from nucleotides or amino acids (both naturally occurring or synthetically made) in large numbers but of course they need not be limited to these. In another aspect, the nucleic acid aptamers are short strands of DNA that bind protein targets. In one aspect, the aptamers are oligonucleotide aptamers. Oligonucleotide aptamers are oligonucleotides which can bind to a specific protein sequence of interest. A general method of identifying aptamers is to start with partially degenerate oligonucleotides, and then simultaneously screen the many thousands of oligonucleotides for the ability to bind to a desired protein. The bound oligonucleotide can be eluted from the protein and sequenced to identify the specific recognition sequence. Transfer of large amounts of a chemically stabilized aptamer into cells can result in specific binding to a polypeptide of interest, thereby blocking its function. (For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)).

As used herein, an antagonist or blocking agent may comprise, without limitation, an antibody, an antigen binding portion thereof or a biosynthetic antibody binding site that binds a particular target protein; an antisense molecule that hybridizes in vivo to a nucleic acid encoding a target protein or a regulatory element associated therewith, or a ribozyme, aptamer, or small molecule that binds to and/or inhibits a target protein, or that binds to and/or inhibits, reduces or otherwise modulates expression of nucleic acid encoding a target protein.

Aptamers offer advantages over other oligonucleotide-based approaches that artificially interfere with target gene function due to their ability to bind protein products of these genes with high affinity and specificity. However, RNA aptamers can be limited in their ability to target intracellular proteins since even nuclease-resistant aptamers do not efficiently enter the intracellular compartments. Moreover, attempts at expressing RNA aptamers within mammalian cells through vector-based approaches have been hampered by the presence of additional flanking sequences in expressed RNA aptamers, which may alter their functional conformation.

The idea of using single-stranded nucleic acids (DNA and RNA aptamers) to target protein molecules is based on the ability of short sequences (20 mers to 80 mers) to fold into unique 3D conformations that enable them to bind targeted proteins with high affinity and specificity. RNA aptamers have been expressed successfully inside eukaryotic cells, such as yeast and multicellular organisms, and have been shown to have inhibitory effects on their targeted proteins in the cellular environment.

In one embodiment, the invention relates to methods and reagents for immunizing and treating a subject with cancer to elicit specific cellular and humoral immune-responses against specific cancer cell antigens, including those cancer cell antigens expressed only in cancer cells and in non-cancer cells normally located in one or more immune-privileged sites or tissues of the individual. The invention provides methods of using specifically prepared immunogen in fresh or lyophilized liposome, proper routes of administration of the immunogen, proper doses of the immunogen, and specific combinations of heterologous immunization including DNA priming in one administration route followed by liposome-mediated protein antigen boost in a different route to tailor the immune responses in respects of enhancing cell mediated immune response, cytokine secretion, humoral immune response, especially skewing T helper responses to be Th1 or a balanced Th1 and Th2 type.

For convenience, immune responses are often described in the present invention as being either “primary” or “secondary” immune responses. A primary immune response, which is also described as a “protective” immune response, refers to an immune response produced in an individual as a result of some initial exposure (e.g., the initial “immunization”) to a particular antigen, e.g., cell surface receptor, or activated integrin receptor. Such an immunization can occur, for example, as the result of some natural exposure to the antigen (for example, from initial infection by some pathogen that exhibits or presents the antigen) or from antigen presented by cancer cells of some tumor in the individual (for example, malignant melanoma). Alternatively, the immunization can occur as a result of vaccinating the individual with a vaccine containing the antigen. For example, the vaccine can be a cancer vaccine comprising one or more antigens from a cancer cell e.g., squamous cell carcinoma.

The tumor cell vaccine can also be modified to express other immune activators such as IL2, and costimulatory molecules, among others.

Another type of anti-tumor vaccine that can be combined with antibodies to a cancer testis antigen is a vaccine prepared from a cancer cell line lysate of interest, in conjunction with an immunological adjuvant, or a mixture of lysates from two human cancer cell lines of interest plus DETOX™. immunological adjuvant. Vaccine treatment can be boosted with anti-cancer-testis antigen antibodies, with or without additional chemotherapeutic treatment.

When used in vivo for therapy, the antibodies of the subject invention are administered to the patient in therapeutically effective amounts (i.e., amounts that have desired therapeutic effect). They will normally be administered parenterally. The dose and dosage regimen will depend upon the degree of the infection, the characteristics of the particular antibody or immunotoxin used, e.g., its therapeutic index, the patient, and the patient's history. Advantageously the antibody or immunotoxin is administered continuously over a period of 1-2 weeks, intravenously to treat cells in the vasculature and subcutaneously and intraperitoneally to treat regional lymph nodes. Optionally, the administration is made during the course of adjunct therapy such as combined cycles of radiation, chemotherapeutic treatment, or administration of tumor necrosis factor, interferon or other cytoprotective or immunomodulatory agent.

For parenteral administration the antibodies will be formulated in a unit dosage injectable form (solution, suspension, emulsion) in association with a pharmaceutically acceptable parenteral vehicle. Such vehicles are inherently nontoxic, and non-therapeutic. Examples of such vehicle are water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability, e.g., buffers and preservatives. The antibodies will typically be formulated in such vehicles at concentrations of about 1 mg/ml to 10 mg/ml.

Use of IgM antibodies can be preferred for certain applications, however IgG molecules by being smaller can be more able than IgM molecules to localize to certain types of infected cells.

There is evidence that complement activation in vivo leads to a variety of biological effects, including the induction of an inflammatory response and the activation of macrophages (Unanue and Benecerraf, Textbook of Immunology, 2nd Edition, Williams & Wilkins, p. 218 (1984)). The increased vasodilation accompanying inflammation can increase the ability of various agents to localize in infected cells. Therefore, antigen-antibody combinations of the type specified by this invention can be used therapeutically in many ways. Additionally, purified antigens (Hakomori, Ann. Rev. Immunol. 2:103, 1984) or anti-idiotypic antibodies (Nepom et al., Proc. Natl. Acad. Sci. USA 81: 2864, 1985; Koprowski et al., Proc. Natl. Acad. Sci. USA 81: 216, 1984) relating to such antigens could be used to induce an active immune response in human patients. Such a response includes the formation of antibodies capable of activating human complement and mediating ADCC and by such mechanisms cause infected cell destruction.

Optionally, the antibodies of this invention are useful as antibody-cytotoxin conjugate molecules, as exemplified by the administration for treatment of neoplastic disease.

The antibody compositions used in therapy are formulated and dosages established in a fashion consistent with good medical practice taking into account the disorder to be treated, the condition of the individual patient, the site of delivery of the composition, the method of administration and other factors known to practitioners. The antibody compositions are prepared for administration according to the description of preparation of polypeptides for administration, infra.

As is well understood in the art, biospecific capture reagents include antibodies, binding fragments of antibodies which bind to activated integrin receptors on metastatic cells (e.g., single chain antibodies, Fab′ fragments, F(ab)′₂ fragments, and scFv proteins and affibodies (Affibody, Teknikringen 30, floor 6, Box 700 04, Stockholm SE-10044, Sweden; See U.S. Pat. No. 5,831,012, incorporated herein by reference in its entirety and for all purposes)). Depending on intended use, they also can include receptors and other proteins that specifically bind another biomolecule.

The hybrid antibodies and hybrid antibody fragments include complete antibody molecules having full length heavy and light chains, or any fragment thereof, such as Fab, Fab′, F(ab′)₂, Fd, scFv, antibody light chains and antibody heavy chains. Chimeric antibodies which have variable regions as described herein and constant regions from various species are also suitable. See for example, U.S. Application No. 20030022244.

Initially, a predetermined target object is chosen to which an antibody can be raised. Techniques for generating monoclonal antibodies directed to target objects are well known to those skilled in the art. Examples of such techniques include, but are not limited to, those involving display libraries, xeno or humab mice, hybridomas, and the like. Target objects include any substance which is capable of exhibiting antigenicity and are usually proteins or protein polysaccharides. Examples include receptors, enzymes, hormones, growth factors, peptides and the like. It should be understood that not only are naturally occurring antibodies suitable for use in accordance with the present disclosure, but engineered antibodies and antibody fragments which are directed to a predetermined object are also suitable.

The present application discloses compositions and methods for inhibiting the proteins described herein, and those not disclosed which are known in the art are encompassed within the invention. For example, various modulators/effectors are known, e.g. antibodies, biologically active nucleic acids, such as antisense molecules, RNAi molecules, or ribozymes, aptamers, peptides or low-molecular weight organic compounds recognizing said polynucleotides or polypeptides.

### EXAMPLES

### Example 1

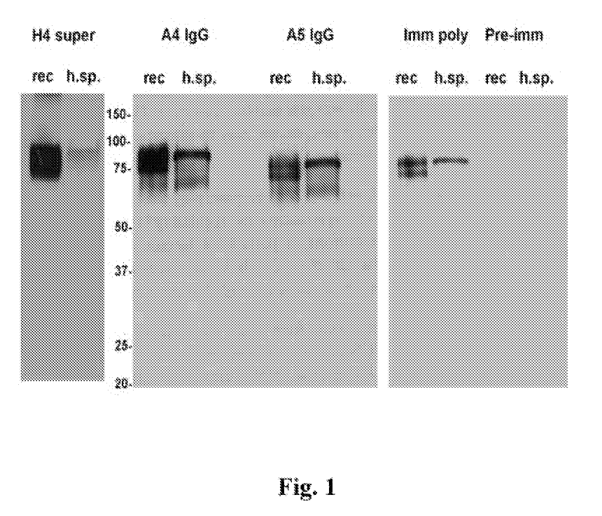

Immunoreagents to CABYR Dr. Herr's group recently developed 3 monoclonal antibodies to CABYR (FIG. 1) which were used to demonstrate CABYR is a biomarker for squamous tumors. These monoclonal immunoreagents were used to detect expression of CABYR peptides on human sperm by immunofluorescence. They reacted only with the sperm principal piece (data not shown), confirming their specificity for the native CABYR protein.

Validation of mAbs as probes to image human CABYR protein in paraffin embedded human tissues. A first step in establishing the utility of these monoclonal antibodies for screening tumor biopsies was to test their staining characteristics on paraffin embedded human testes. Since CABYR expression has been shown to be restricted to post-meiotic cells in the final step of differentiation in the testes (Soren Naaby-Hansen et al, (2002) Dev. Biol. 242: 236-254), the testis sections served as a positive control for testing CABYR immunoreagents in tumors. To optimize these immuno-reagents for initial tumor studies a minimal dilution was identified that gave a strong and specific signal in the testis where CABYR is abundant. A 1:20,000 dilution of a cocktail containing the 3 mAbs was observed to specifically stain only spermatids and luminal spermatozoa in the human seminiferous epithelium (data not shown).

CABYR Appears in a Subset of Human Cancers. The 1:20,000 dilution of mAbs was then utilized to localize CABYR in tumor samples of the head and neck. Since CABYR may be present at lower concentrations in some tumor cells, mAbs were also tested at higher concentrations. An initial immunohistochemical study of a tissue microarray of paraffin embedded specimens containing 101 cases of squamous cell carcinoma of the head & neck and lung was conducted. At a 1:1000 dilution of the cocktail of monoclonal antibodies to CABYR, 22 of 51 cancers of the head and neck were positive (43%). At a dilution of 1:20,000, 7 of the 51 head and neck tumors were positive (14%). When an additional 50 lung tumors were screened at a 1:20,000 dilution, 7 of the 50 were CABYR positive (14%). In a punch biopsy of a lung squamous cell carcinoma CABYR staining at the 1:20,000 dilution was concentrated solely in the squamous tumor cells while being absent in the noncancerous tissue below the collar of leukocytes. Based on this initial tumor screen it was concluded that at a minimum 14% of head & neck tumors express CABYR while the incidence likely will be higher once an optimal dilution for immunohistochemistry is determined. A prior report for lung tumor CABYR positivity was 39% (Chonglin Luo et al, (2007) Clin. Cancer Res 13 (4) 1288-1297).

### Example 2

The Human Testis: Normal Site Where CABYR is Found.

The CABYR gene and CABYR protein were first cloned and characterized in both humans (Soren Naaby-Hansen et al, (2002) Dev. Biol. 242: 236-254) and mice (Buer, Sen et al., 2003, Gene 310:67-78). The single copy CABYR gene was named to signify: a calcium binding protein that is tyrosine phosphorylation-regulated. The CABYR protein was identified during human development to arise first at puberty in the testis with the onset of spermatogenesis. CABYR mRNAs have been noted to be translated following meiosis only in round spermatids. Thus, the protein appears at the last step of sperm differentiation. The CABYR protein localized to a specific domain of the sperm flagellum known as the principal piece, where CABYR was found in the ribs and longitudinal columns of the fibrous sheath, a cytoskeletal structure known to be a scaffold where glycolytic enzymes are organized.

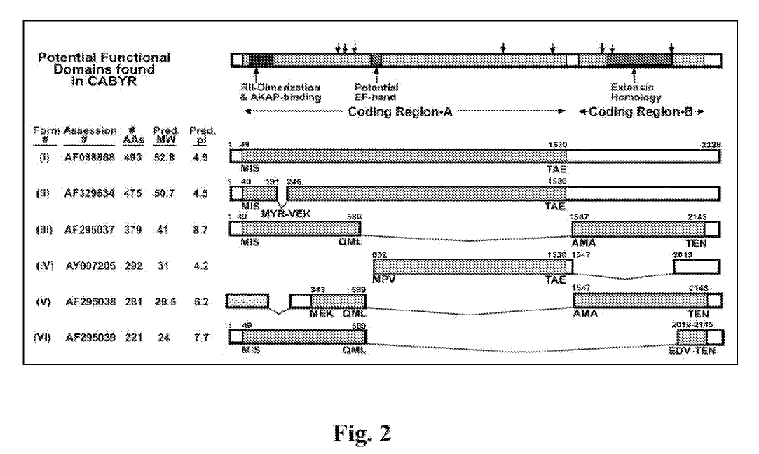

The CABYR gene undergoes alternative splicing to produce 6 distinct proteins. Six alternative splice variants of CABYR are translated in spermatids resulting in considerable CABYR protein microheterogeneity. The six isoforms contain different functional domains. Various CABYR isoforms in tumors will be examined by RT PCR. Knowing which isoforms are present in tumors will insure the correct monoclonal antibodies are identified for clinical assay development and suggest drug development opportunities. The human genome has a single functional copy of CABYR on chromosome 18 segregated into 6 CABYR exons spanning 18.5 kb of genomic DNA. The CABYR gene is organized into two distinct coding regions, designated A & B (CR-A & CR-B, see FIG. 2). A stop codon at the end of CR-A is separated by 15 in-frame bases from the start codon in CR-B. As shown in FIG. 2, the functional domains in CABYR are blocked, including putative RII dimerization and AKAP binding and extensin motifs. Arrowheads above the figure denote PXXP motifs in human CR-A and CR-B. On Northern blots two broad mRNA bands of approximately 2.4 and 1.4 Kb were noted in human testicular mRNAs, indicating that multiple CABYR messages of different sizes are transcribed. The six splice variants, I-VI, cloned by the PI to date from the human testis represent all GenBank EST deposits for CABYR. Recombinant forms of human CABYR have been expressed and used to determine that the coding region A is responsible for calcium binding. These recombinant CABYR proteins will be used as targets to detect anti-CABYR antibodies in patients with CABYR positive tumors.

To identify the expression of CABYR splice variants in cancerous lung tissue from lung cancer patients, variant specific primers were designed flanking the spliced region of the variant and PCR analyses were performed on the cDNAs amplified from RNA in patient's lung cancer biopsies. Several CABYR variants were expressed in lung cancer tissue biopsies. Isoform 3 (SEQ ID NO: 5) was abundantly expressed in lung cancer tissues (88%; 14/16, squamous 100% vs adenocarcinoma 71% ), isoform 1 (SEQ ID NO: 1) was present in 25% (4/16, squamous 22% vs adenocarcinomas 28%), isoform 5 (SEQ ID NO: 5) was expressed in 56% (9/16, squamous 44% vs adenocarcinomas 71%) and isoform 6 (SEQ ID NO: 6) was expressed in 56% (squamous 67% vs adenocarcinomas 43%), isoform 2 (SEQ ID NO: 2) was expressed in 37% (6/16, squamous 44% vs adenocarcinomas 28%) while isoform 4 (SEQ ID NO: 4) was not detected in any sample. Human lung cancer squamous cell carcinoma line H226 showed a similar pattern of CABYR expression to that found in tumor biopsies with relatively increased expression of isoform 3 compared to the other isoforms. Western blot analysis of 5 lung tumor samples also revealed expression of a major CABYR protein at ˜77 kDa size and two minor bands of ˜65 kDa and ˜50 kDa (80% of samples) which were also noted in human spermatozoa. Ten cancerous cell lines including squamous (5 from head and neck tumors), adenocarcinomas (2) and other cancer cell lines (3) were examined for CABYR expression. All the cell lines showed CABYR protein expression with three major bands of ˜77 kDa, ˜65 kDa and 50 ˜kDa. Additionally, three minor bands were noted between 77 kDa and 65 kDa. Two dimensional SDS-PAGE analyses of H226 cell protein extracts showed ˜20 immuno-reactive spots within the pI range from ˜6.5-7.5 using two anti-hCABYR monoclonal antibodies. Squamous cell lung cancer line H226 was observed to contain mainly isoforms between pI 6-7, likely those containing both CRA and CRB. This is consistent with the variant 3 peptide (SEQ ID NO: 6) being predominant in tumors and tumor cell lines.

Several CABYR protein isoforms were seen in the neutral to alkaline side while human spermatozoa CABYR proteins were predominantly in the acidic range (pI ˜4.5-5.5). This suggests CABYR may undergo post-translational modifications in tumor cells that differ from those occurring during spermatogenesis. CABYR localization in human lung cancer tissue sections was confirmed by using anti-hCABYR antibody. Immunohistochemistry of lung tissue showed that CABYR was localized in the cytoplasm of cells in the cancerous part of lung tissue but not in the normal region. Immunofluorescence localization of CABYR in different cancer cell lines showed cytoplasmic localization. H226, a human lung cancer squamous cell line also showed cytoplasmic localization.

CABYR in Human Cancer. In addition to GenBank deposits from the human testis, there were 4 CABYR hits for lung in the current NCBI data base of ESTs (expressed sequence tags)—all these ESTs were from human lung tumors. These data indicated that, in addition to normal expression in spermatids and sperm, CABYR was expressed in lung tumors. Applicants conducted RT-PCR studies with primers specific for the 6 CABYR splice variants to determine which forms of CABYR were present in human tumor samples. A cohort of 16 lung cancer specimens were studied in which the tumors had been divided under the dissecting microscope into cancerous and “noncancerous” regions and each region was dissected to isolate RNA while also fixing a portion for histology. The patients were all smokers. Seven of these tumors were typed by the pathologists as adenocarcinomas and 9 were designated squamous cell carcinomas. FIG. 3 shows the results from this PCR study.

CABYR mRNAs were identified in 100% of squamous cell lung tumors. CABYR variant I was noted in 25% (4/16) of patients, including 2 of the 7 adenocarcinomas and 2/9 squamous cell carcinomas. Variant II was noted in 37% (6/16 patients), 2/7 adenocarcinomas, 4/9 squamous cell. CABYR variant III was found to be expressed in 100% of squamous cell lung cancers [9/9] and in 71% [5/7] of adenocarcinomas. Variant IV was absent in all tumors tested. Variant V was detected in 56% (9/16) of patients, 5 adenocarcinomas, 4 squamous. Variant VI was detected in 56% (9/16), 2/7 being adenocarcinomas and 6/9 being squamous.

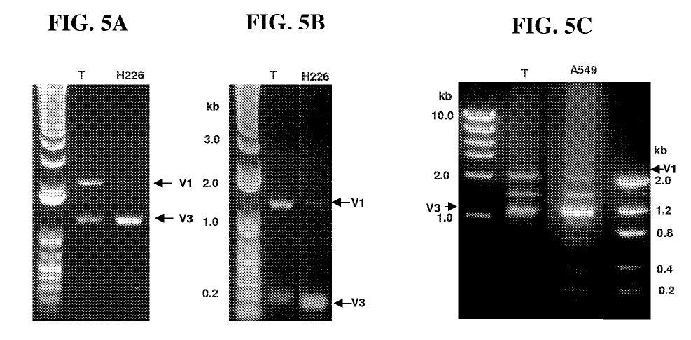

As shown in FIGS. 5A-C, RT -PCR analysis of CABYR mRNA expression in lung cancer cell lines H226 and A549 was compared to native testicular expression. As shown in FIG. 5A, in human testes variant 1 and variant 3 were expressed at roughly equal levels, while in the tumor cell line H226 variant 3 was the dominant isoform. Using an alternative set of primers for variant 3, the results were duplicated revealing approximately equal amounts of variant 1 and variant 3 in human testis, while variant 3 predominated in the tumor cell line (see FIG. 5B). As shown in FIG. 5C, variant 1 specific amplification in testis and A549 reveals V3 being most abundant in A549 while in testis V1 and V3 were almost of equal density. H266 is a squamous cell lung cancer line while A549 is a adenocarcinoma lung cancer line. The observations demonstrate that differential expression of CABYR isoforms occurs in lung cancer cell lines and in lung tumors. CABYR variant 3 is expressed at higher levels in the tumor cell lines than in testis and therefore diagnostics based on this variant may provide a more reliable diagnostic marker for cancer.

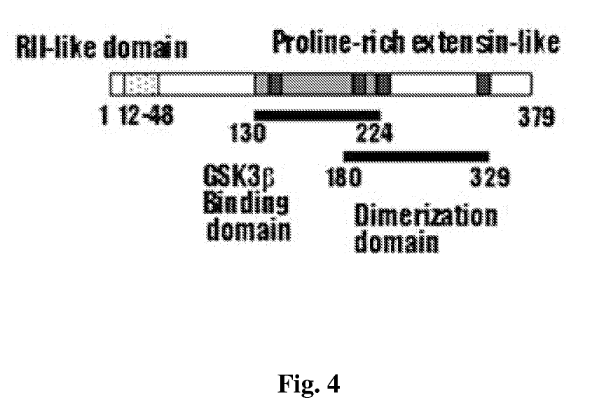

Importance of pilot study. This result shows that the CABYR gene is expressed in all squamous lung tumors and in many lung adenocarcinomas. It reveals that of the 6 possible CABYR isoforms, the CABYR variant III is present in every tumor. The schematic drawing of FIG. 4 summarizes the functional domains in CABYR isoform III. The fact that preliminary evidence indicates that variant III is present in 100% of squamous cell cancers is exciting because this variant contains the potentially drugable domains of RII dimerization (this mediates AKAP binding), GSKβ binding and CABYR dimerization. One of the most significant results of this study was the observation that each of the CABYR isoforms amplified was either absent or at very low levels in the “noncancerous” portion of the surgical mass levels. This result provides a compelling argument that CABYR expression is specific to tumor cells.

CABYR is Immunogenic In Humans

To determine if CABYR is a human autoantigen, anti-sperm antibodies from infertile men were evaluated for CABYR immunoreactivity. 14% of samples from infertile ASA+ men had anti-CABYR antibodies.

In summary: CABYR mRNAs and proteins were detected in squamous cell lung cancers and in adenocarcinomas. Men with antisperm antibodies (ASA) recognize CABYR suggesting the molecule is immunogenic in humans and might be a target for immunotherapy for lung cancer patients.

### Conclusion

Among normal tissues, CABYR is a calcium binding protein expressed during spermatogenesis specifically in round and elongated spermatids. In patients with anti-sperm antibodies, CABYR immunoreactivity was noted in approximately 14% indicating the molecule is auto-immunogenic in humans. CABYR mRNAs were expressed in all human lung cancer tissues. Isoform 3 was expressed in 100% of squamous cell carcinomas. CABYR transcripts were also noted in lung cancer cell line (H226). Increased expression of isoform 3 was seen relative to other isoforms in this cell line. CABYR protein (˜77 kDa, 65 kDa & 50 kDa) bands were observed in human lung cancer tissues as well as cell lines. Two-dimensional analysis of cell line H226 showed ˜20 immunoreactive spots, predominantly in the neutral range (pI 6.0-7.0). Immunofluorescence localization of CABYR in human squamous cell lung cancer tissue biopsies as well as a cell line showed cytoplasmic localization. The normal restricted expression of CABYR to the human testis suggests that in tumors that express this protein, CABYR might offer a selective target suitable for both diagnosing squamous tumors or directing drugs or therapeutic cancer-vaccines.

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

1. A method of screening for squamous cell cancer in a subject, said method comprising detecting the expression of the sperm flagellar protein, CABYR, in a biological sample of said subject.

2. The method of claim 1 wherein the individual expression of CABYR variant III is detected.

3. The method of claim 1 wherein the expression of CABYR is detected by analyzing a biological sample of said subject for the presence of RNA transcripts of the CABYR gene.

4. The method of claim 3 wherein said RNA transcript comprises a sequence of SEQ ID NO: 5 that is not present in any of the sequence of SEQ ID NO: 1, SEQ ID NO: 3, SEQ ID NO: 7, SEQ ID NO: 9 and SEQ ID NO: 11.

5. The method of claim 3 wherein the expression of CABYR is detected by an RT-PCR assay.

6. The method of claim 1 wherein the expression of CABYR is detected by analyzing a biological sample of said subject for the presence of a peptide sequence comprising at least 6 amino acids identical a sequence selected from the group consisting of SEQ ID NO: 2, SEQ ID NO: 4, SEQ ID NO: 6, SEQ ID NO: 8, SEQ ID NO: 10 and SEQ ID NO: 12.

7. The method of claim 6 wherein the expression of CABYR is detected by an immunoassay that uses an antibody that specifically binds to a peptide selected from the group consisting of SEQ ID NO: 2, SEQ ID NO: 4, SEQ ID NO: 6, SEQ ID NO: 10 and SEQ ID NO: 12.

8. The method of claim 7 wherein the antibody specifically binds to a peptide selected from the group consisting of SEQ ID NO: 6, SEQ ID NO: 10 and SEQ ID NO: 12.

9. The method of claim 8 wherein the antibody specifically binds to a peptide of SEQ ID NO: 6.

10. A method of monitoring a cancer treatment for squamous cell cancer in a subject, said method comprising
detecting the expression of the sperm flagellar protein, CABYR, in a biological sample of said subject prior to initiating said treatment;
continuing to monitor the expression of the sperm flagellar protein, CABYR through out said treatment, wherein a decreased level of expression is indicative of an effective treatment.

11. An antibody capable of binding specifically to a polypeptide of SEQ ID NO 6.

12. The antibody of claim 11, wherein the antibody is a monoclonal antibody, a bispecific antibody, a chimeric antibody, or a humanised antibody.

13. The antibody of claim 11, wherein the antibody is conjugated to a therapeutic moiety, said therapeutic moiety selected from the group consisting of a second antibody or a fragment or derivative thereof, a cytotoxic agent and a cytokine.
